(19) United States
(12) Patent Application Publication
(10) Pub. No.: US 2017/0072034 A1
(43) Pub. Date: Mar. 16, 2017

(54) THERAPEUTIC CANCER VACCINE TARGETED TO HAAH (ASPARTYL-[ASPARAGINYL]-BETA-HYDROXYLASE)

(71) Applicant: Panacea Pharmaceuticals Inc., Gaithersburg, MD (US)

(72) Inventors: Biswajit BISWAS, Germantown, MD (US); Carl R. MERRIL, Bethesda, MD (US); Hossein A. GHANBARI, Potomac, MD (US)

(73) Assignee: Panacea Pharmaceuticals Inc., Gaithersburg, MD (US)

(21) Appl. No.: 15/176,752

(22) Filed: Jun. 8, 2016

Related U.S. Application Data
(63) Continuation-in-part of application No. 13/836,487, filed on Mar. 15, 2013, now abandoned.

(51) Int. Cl. A61K 39/00; C12N 9/02

(52) U.S. Cl. CPC A61K 39/0011; C12N 9/0071; C12Y 114/11016

(57) ABSTRACT

The present invention encompasses a cancer vaccine therapy targeting Aspartyl-[Asparaginyl]-beta.-hydroxylase (HAAH). The present invention contemplates bacteriophage expressing HAAH peptide fragments and methods for using said bacteriophage in methods of treating cancer.

This application is a Continuation-In-Part of U.S. application Ser. No. 13/836,487, filed Mar. 15, 2013 of which are incorporated herein by reference in their entirety.

### BACKGROUND OF THE INVENTION

Cancer is one of the most devastating diseases both in terms of human life opportunity loss and health care cost. It also presents unmet clinical needs. Currently available chemotherapies have limited efficacy and limited target patient population. Even the successful immunotherapies have shortcomings similar to chemotherapies. Moreover, essentially all cancer therapeutics have significant adverse side effects.

Aspartyl-(Asparaginyl)-β-hydroxylase (HAAH) is over expressed in various malignant neoplasms, including hepatocellular and lung carcinomas. HAAH is a tumor specific antigen, which is specifically expressed on the surface of certain malignant cells. HAAH is a hydroxylation enzyme that modifies factors such as Notch that contribute to cancer etiology by causing cell proliferation, motility, and invasiveness. Neutralizing the enzyme or reducing its expression leads to normal phenotype(s) in cancer cells. Anti-HAAH antibodies (as well as siRNA) have been shown to be cytostatic. An all-human sequence anti-HAAH (PAN-622) has shown to inhibit tumor growth by more than 90% in animal studies by passive immunotherapy. However, HAAH is well conserved and is also over expressed in placenta hence it is not sufficiently immunogenic in animals and it is certainly a self antigen in humans.

A vaccine therapy targeted to a pan-cancer-specific antigen such as HAAH that has proven relevance to cancer etiology is very desirable. Its economic impact will be enormous both in terms of job creation and increased productivity as well as in savings in health care and extending productive lives. The vaccine therapy of the present invention is novel both in terms of its target and the vaccine entity.

### SUMMARY OF THE INVENTION

The present invention encompasses a cancer vaccine therapy targeting human Aspartyl-[Asparaginyl]-β-hydroxylase (HAAH).

Certain embodiments of the present invention contemplate bacteriophage expressing HAAH peptide fragments, wherein the bacteriophage may be any one of Lambda, T4, T7, or M13/f1.

The present invention further contemplates methods of treating cancer comprising stimulating the immune system of a patient with bacteriophage expressing HAAH fragments.

The present invention also contemplates nano-particles comprising at least one amino acid sequence native to HAAH.

The present invention also encompasses methods for treating cancer comprising the step of providing an immune system stimulating amount of a Lambda phage to a patient, wherein the Lambda phage comprises amino acid sequences native to HAAH expressed on its surface.

The present invention also encompasses methods for treating cancer comprising the step of providing an immune system-stimulating amount of a nano-particle to a patient, wherein the nano-particle comprises amino acid sequences native to HAAH.

One embodiment of the present invention contemplates bacteriophage comprising at least one amino acid sequence native to HAAH, wherein the at least one amino acid sequence native to HAAH is selected from the group consisting of the amino acid sequence of Construct I, the amino acid sequence of Construct II and the amino acid sequence of Construct III.

The present invention also contemplates a Lambda phage expressing the amino acid sequence of Construct I, the amino acid sequence of Construct II or the amino acid sequence of Construct III on its surface.

Embodiments of the present invention also contemplate nucleic acid construct comprising at least one nucleotide sequence encoding an amino acid sequence native to HAAH and a nucleic acid sequence encoding bacteriophage lambda head decoration protein D (hereinafter “gpD”).

Another embodiment of the present invention includes nucleic acid constructs comprising nucleotide sequences encoding the amino acid sequence of Construct I, the amino acid sequence of Construct II or the amino acid sequence of Construct III.

### BRIEF DESCRIPTION OF THE FIGURES

FIG. 1 is a graph that demonstrates the efficacy of an antibody against HAAH in live cancer cells.

FIG. 2 shows the mechanism of immunization in accordance with the present invention.

FIG. 3 shows the immune response.

FIG. 4 Homologous recombination of donor plasmid pAN-A- with recipient phage vector. Only some of the lambda genes are shown. The unique Nhe I and Bssh II site in the lambda genome used for cloning is shown as is lacZa, a DNA cassette comprised of lacPO, RBS and the first 58 codons of lacZ. Generated recombinant phages are designated as HAAH construct I, II and III which contains an insert of HAAH fragment. Only diagram of construct I is shown here. The insert is fused with gpD head protein gene of lambda to produce gpD-HAAH construct I fusion on lambda capsid. The maps are not to scale.

FIG. 5 shows an example example of a Western blot HAAH-vaccine screening for a cancer vaccine candidate.

FIG. 6 is a scatter chart showings the results of HAAH tests as a cancer biomarker on a group of 857 individuals composed of 211 individuals known not to have cancer and 646 patients who are diagnosed with cancer. The cancer group is composed of a mix of individuals with different types of cancer (Breast, Prostate, Lung, Colon) in various stages from one to four. Combining the 12 false positive and 34 false negative results, the test has less than 5.4% error even in such a large group of patients. Horizontal axis is the patient index.

FIG. 7 shows amino acid sequences in accordance with an embodiment of the present invention.

FIG. 8 shows the amino acid sequence of the GpD-HAAH-1λ fusion protein. The GpD sequence is highlighted in blue and the HAAH sequence is highlighted in green.

FIG. 9 Analysis of HAAH Lambda Constructs for HAAH-1λ
Left panel: Western blot of HAAH constructs 1, 2, 3 and parental phage pVCDCDL3 plaques
Right panel: Western blot of HAAH constructs 1, 2, 3 and parental phage pVCDCDL3 from SDS-PAGE. Both blots are probed with FB50 monoclonal antibody that is specific to the N-terminal portion of HAAH.

FIG. 10 is a flow diagram of an exemplary manufacturing process for HAAH-1λ.

FIG. 11 shows ELISA data for immunogencity of Drug Product: rHAAH-coated plates.

FIG. 12 shows ELISA data for immunogenicity of Drug Product: HAAH-1λ-coated plates.

FIG. 13 shows ELISA data for immunogenicity of PAN-301-1Drug Product in rats: rHAAH-coated plates.

FIG. 14 shows ELISA data for immunogenicity of PAN-301-1Drug Product in rats: HAAH-1λ-coated plates.

FIG. 15 shows the results of a cytotoxicity assay comparing the use of spleen cells from HAAH-1λ(phage)-vaccinated or control mice.

FIG. 16 shows ELISA testing of mouse sera from animals injected either intramuscularly or intradermally. Plates are coated with recombinant HAAH (blue bars) or the human lung cancer cell line, H460 (green bars). Sera tested were obtained on Days 14, 28 and 35.

FIG. 17 shows flow cytometry analysis of binding of anti-HAAH monoclonal antibodies to human, mouse and rat tumor cell lines.

FIG. 18 shows a treatment tumor challenge study in mice with liver tumor cell line BNLT3. Tumor volume is calculated as follows: Tumor volume (mm³)=length×width ×0.5. *p<0.05.

FIG. 19 shows a tumor challenge study with the BNLT3 liver cancer cell line. Tumors were excised upon sacrifice and weighed. The HAAH-1λ-vaccinated group is labeled “phage”.

FIG. 20 shows a tumor challenge model using mouse breast cancer cell line, 4T1. Tumor volume is calculated as follows: Tumor volume (mm³)=length×width²×0.5. *p<0.05.

FIG. 21 shows a tumor challenge study in mice using the breast cancer cell line 4T1. The number of metastases in the lungs were determined upon sacrifice. **p<0.01, ***p<0.001.

FIG. 22 shows survival curves in a mouse challenge study using the breast cancer cell line 4T1.

FIG. 23 shows metastasis of the rat prostate tumor cell line MLLB-2, observed as hind limb paralysis.

### DETAILED DESCRIPTION OF THE INVENTION

For simplicity and illustrative purposes, the principles of the present invention are described by referring to various exemplary embodiments thereof Although the preferred embodiments of the invention are particularly disclosed herein, one of ordinary skill in the art will readily recognize that the same principles are equally applicable to, and can be implemented in other systems, and that any such variation would be within such modifications that do not part from the scope of the present invention. Before explaining the disclosed embodiments of the present invention in detail, it is to be understood that the invention is not limited in its application to the details of any particular arrangement shown, since the invention is capable of other embodiments. The terminology used herein is for the purpose of description and not of limitation. Further, although certain methods are described with reference to certain steps that are presented herein in certain order, in many instances, these steps may be performed in any order as would be appreciated by one skilled in the art, and the methods are not limited to the particular arrangement of steps disclosed herein.

The present invention is based on the discovery that bacteriophage surface-expressed HAAH is highly immunogenic and could overcome tolerance of self antigen because of altered presentation and the adjuvant function of bacteriophage itself. The present invention provides a cancer vaccine therapy targeting HAAH using bacteriophage-expressed HAAH fragments.

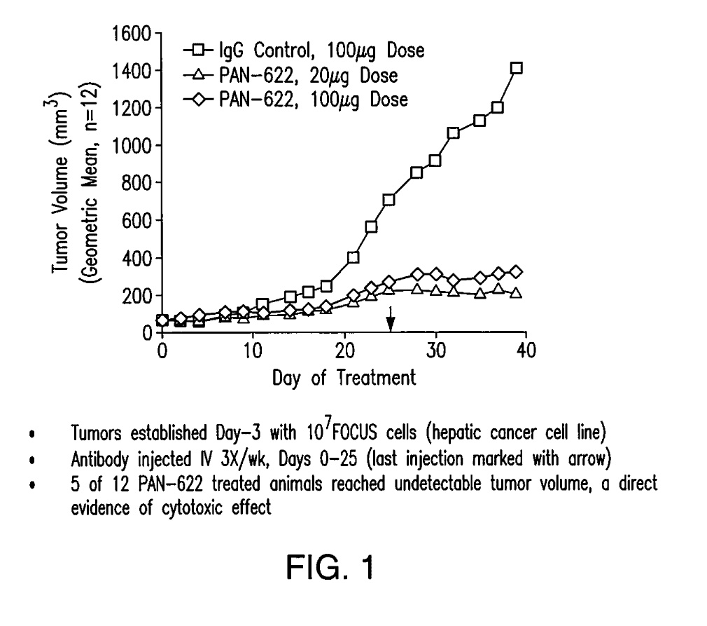

It has been shown that passive immunotherapy using an all-human anti-HAAH is effective in cellular and animals models of cancer (in nude mice model, FIG. 1). The present invention demonstrates that bacteriophage delivery of HAAH fragments as vaccine can overcome the problem of self antigen tolerance by providing novel antigen presentation and inherent phage adjuvant properties.

In vitro activation of dendritic cells by tumor antigens, prior to administration to patient body shows promising results for cancer therapy. Unfortunately the process is cumbersome, expensive and time consuming for mass scale immune therapy against various cancers. Bacteriophage display is a simple way of achieving favorable presentation of peptides to the immune system. Previous findings revealed that recombinant bacteriophage can prime strong CD8+ T lymphocytes (CTLs) responses both in vitro and in vivo against epitopes displayed in multiple copies on their surface, activate T-helper cells and elicit the production of specific antibodies all normally without adjuvant.

As proposed herein, vaccination with lambda phage-displaying cancer specific antigen such as HAAH has a number of potential advantages. One of the advantages is display of multiple copies of peptides on the same lambda phage, and once the initial phage display has been made, subsequent production should be far easier and cheaper than the ongoing process of coupling peptides to carriers. There is also good evidence that due to particulate nature, phage-displayed peptides can access both the major histocompatibility complex (MHC) I and MEW II pathway, suggesting lambda phage display vaccines can stimulate both cellular and humoral arms of the immune system, although as extra cellular antigens, it is to be expected that the majority of the responses will be antibody (MHC class II) biased. It has been shown that particulate antigens, and phage in particular, can access the MHC I pathway through cross priming, and it is likely that it is this process which is responsible for stimulating a cellular response. This added cellular response mediated by CD8+ T cells helps to eliminate the cancer cells. Also, the role of Innate immunity in cancer is well established fact. Lambda phage can also act as nonspecific immune stimulators. It is likely that a combination of the foreign DNA (possibly due to the presence of CpG motifs) and the repeating peptide motif of the phage coat are responsible for the nonspecific immune stimulation. As a summary: whole lambda phage particles possess numerous intrinsic characteristics which make them ideal as vaccine delivery vehicles. For use as phage display vaccines, the particulate nature of phage means they should be far easier and cheaper to purify than soluble recombinant proteins since a simple centrifugation/ultra-filtration and column chromatography step should be sufficient to remove the majority of soluble contaminants. Additionally, the peptide antigen comes already covalently conjugated to an insoluble immunogenic carrier with natural adjuvant properties, without the need for complex chemical conjugation and downstream purification processes which must be repeated with each vaccine batch.

The present invention provides a prophylactic and therapeutic “phage vaccine” for both cancer prevention and treatment. In the present invention, fragmented HAAH peptides are successfully displayed on the surface of lambda head and large scale production and purification is carried out to perform animal experiments. The detail of these procedures is depicted below.

### A. Construction of Bacteriophage Lambda for Display of HAAH Peptides:

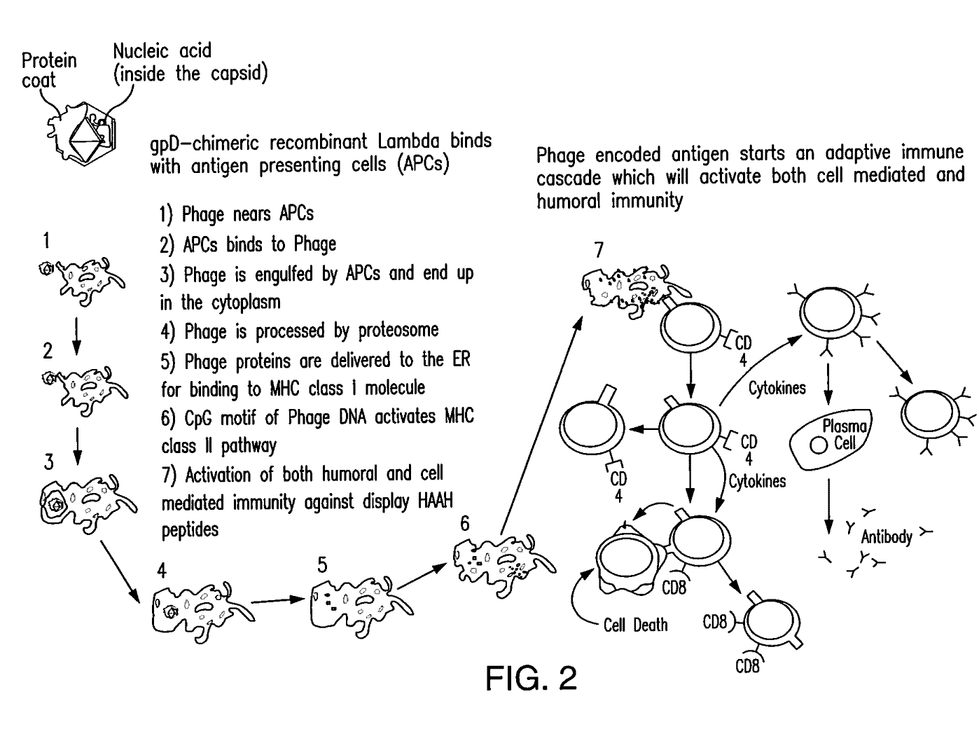

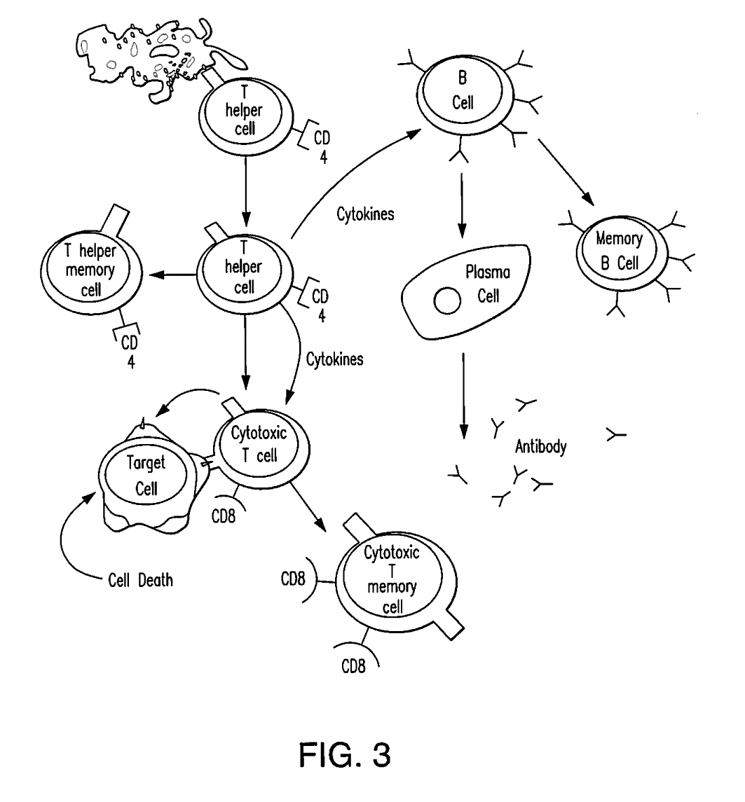

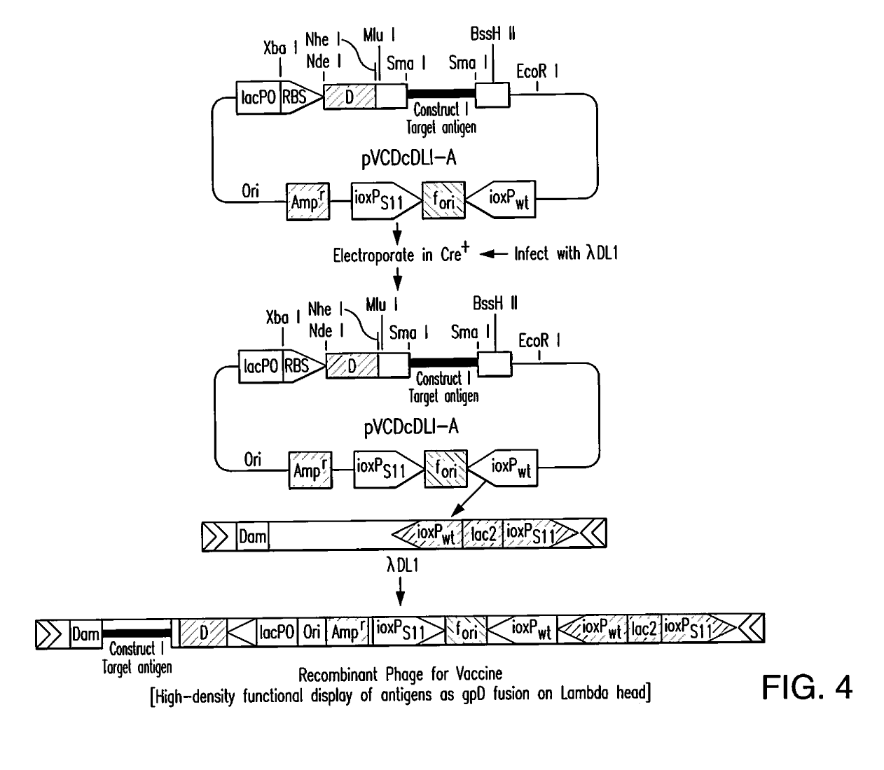

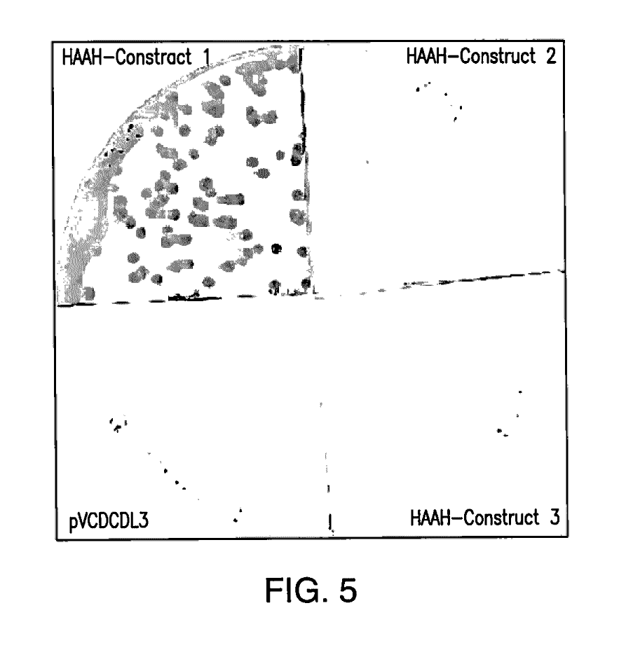

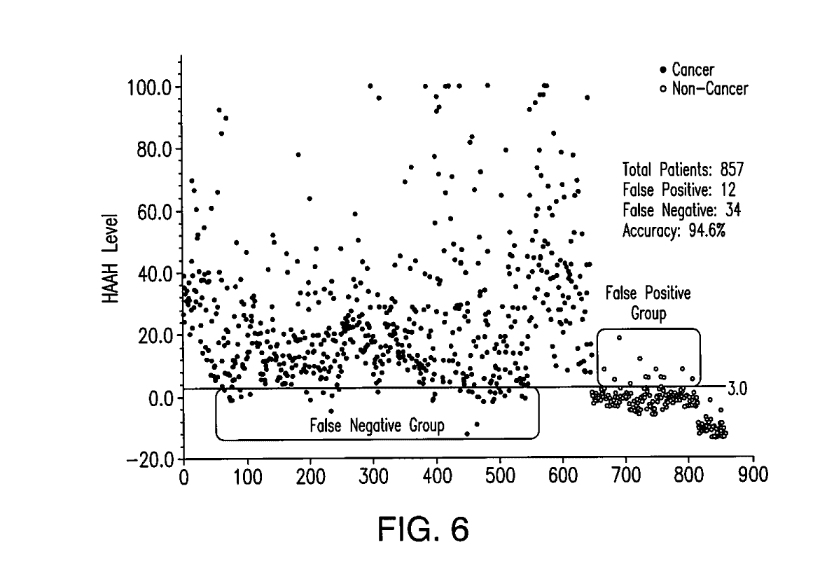

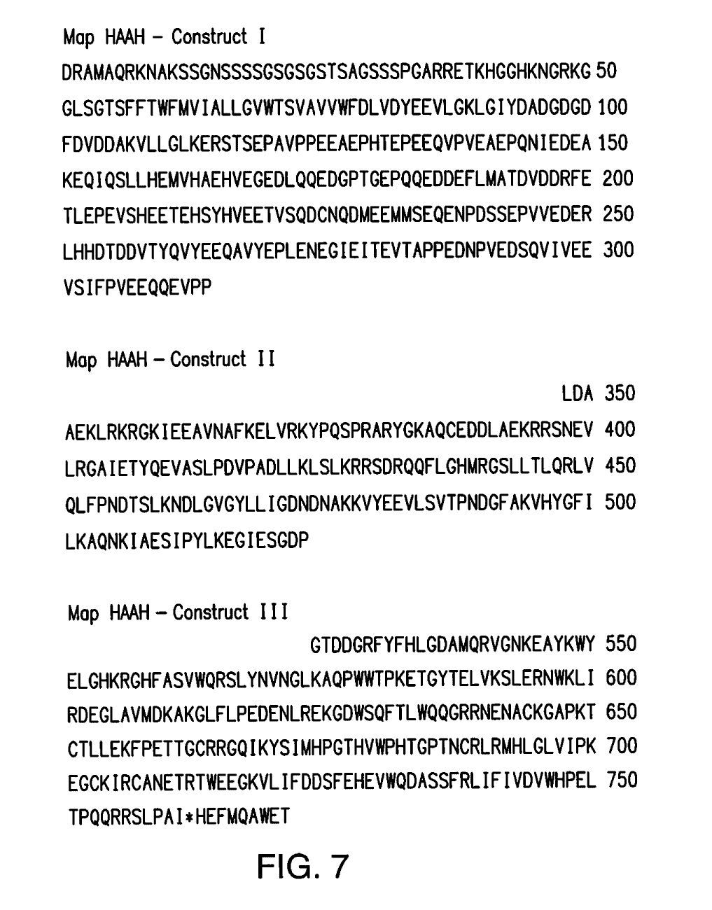

We designed a bacteriophage lambda system to display HAAH peptides fused at the C terminus of the head protein gpD of phage lambda. Molecular analysis of HAAH reveals a partial amino terminal homology of this protein with other two proteins called Junctin and Humbug. The role of these other two proteins in human physiology is not known completely. To avoid any complication such as activating immune system against these homologous proteins, we specifically eliminated these sequences from our phage display constructs. For proper display of HAAH peptides on lambda head, the rest of the HAAH sequence is segmented in three sections. They are designated as HAAH construct 1, HAAH construct 2 and HAAH construct 3 (see FIG. 7). Using HAAH specific oligo primers these segments are amplified from the HAAH gene which was previously cloned in our laboratory for expression in baculovirus system. The oligo sequence of each PCR primer is modified slightly to produce Nhe I and Bssh II restriction sites in each end of amplified HAAH segments. After restriction digestion, these segments are inserted separately at the NheI-BsshII site of the 3′ end of a DNA segment encoding gpD under the control of the lac promoter. The constructs are created in a plasmid vector (donor plasmid pAN-A), which also carries loxPwt and loxP511 sequences. Cre-expressing cells (E. coli) are transformed with these recombinant plasmids and subsequently infected with a recipient lambda phage that carries a stuffer DNA segment flanked by loxPwt and loxP511 sites. Recombination occurs in vivo at the lox sites and Ampr cointegrates are formed (FIG. 2), which are spontaneously lyse the E. coli and released in culture media. The cointegrates produce recombinant phages that display HAAH peptides fused at the C terminus of gpD. Approximately 200 copies of these peptides are displayed on a single phage head.

B. Selection of Lambda Cointegrates and Production of Recombinant Phages which Display HAAH Peptides:

Lambda cointegrates are selected on Luria Bartani (LB) ampicillin agar (100 ug/ml amp, 15% agar) plates. Briefly, spontaneously lysed E. coli culture is used to infect Cre-ve E. coli cells and spread on LB ampicillin agar plates. Plates are incubated at 32° C. for 48 hours to obtain Ampr colonies. These Ampr colonies are immune to super infection and carry the phages as plasmid cointegrates. The Ampr colonies containing the lambda cointegrate are grown separately at LB Ampicillin (100 ug/ml) at 37° C. for four hours. Lambda phages are spontaneously induced in these cultures and result in complete lysis. This cell free supernatant is used to infect E. coli cells and plated on solid LB agar (15%) plate to obtain phage plaques. The resulting phage plaques are harvested from the plate and single plaques are purified three times on E. coli by the standard procedures described by Sambrook et al.

### C. Confirmation of Lambda Cointegrates Containing HAAH Fragments:

All bacterial colonies, containing lambda cointegrates, which are used for HAAH phage vaccine production are verified by PCR. In this process the presence of each cloned inserts in bacterial colonies are confirmed by PCR amplification of HAAH specific insert DNA by XbaI-5/(TTGGTTAGCAAGTTAATACC) and XbaI-3/(TAGATTTGAATGACTTCCCC) primer set.

These two specific primers flank the unique Xba I site of lambda genome and used for PCR the complete insert presence in between Lox recombination sites of lambda DNA.

### D. Growth and Purification of Recombinant Phages Displaying HAAH Peptides:

Growth of the plaque purified phages is performed in two steps. The steps are designated as plate lysate method and large scale liquid lysate method. The detail of these procedures are described in Sambrook et al. The lysed culture is chilled at room temperature for further purification by liquid column chromatography technique.

### E. Large Scale Purification of Recombinant Lambda-Constructs Using Column Chromatography Technique:

CIM® monolithic columns are an ideal chromatographic support for purifying large biomolecules and nanoparticles, bacterial viruses and plasmid DNA. The pore size of these monolithic columns are adjusted to accommodate even the largest molecules and optimized for very high binding capacities at the highest flow rates. We adopted these monolithic columns for large scale purification of lambda phages displaying HAAH-peptides. In order to obtain infective virus during purification process we investigated chemical conditions that provided the maximal yield of phage and which also preserved high infectivity. This information is necessary to adjust chromatographic methods accordingly to avoid undesired phage deactivation during processing.

HPLC equipment: All experiment is performed on a gradient AKTA purifier FPLC chromatography system (GE Healthcare) equipped with Unicorn 5.1 chromatography software, P-900 FPLC pumps, UPC-900 variable wavelength detector, and FRAC-920 fraction collector. CIM ion exchange chromatography is monitored for UV at 280 nm as well as for conductivity and the gradient profile, associated with marks for point of injection and fraction number. Stationary phase: A strong anion exchange (quaternary amine-QA) methacrylate-based CIM disk monolithic column (BIA Separations, Ljubljana, Slovenia) is used for this purification procedure. Mobile phase: 125 mM NaH2PO4, pH 7.0 (loading buffer) and 125 mM NaH2PO4, 1.5 M NaCl, pH 7.0 (elution buffer) of different pH values is used. All buffers is filtered through 0.22 micron pore size filters before use. These strong anion exchange (quaternary amine-QA) methacrylate-based CIM disk monolithic columns is periodically sanitized after processing, by a 2 hour procedure using 1 M NaOH. Processing of phage lysate for QA column analysis: Phage lysates (10 mL) are centrifuged at 12000×g for 10 minutes at 4° C. and the phage containing supernatant is filtered through a 0.22 micron filter prior to loading the phage on the column for chromatography. Collected fractions of 1 mL are analyzed via plaque assay to determine presence of infective phage. Plaque assay data is analyzed to optimize specific conditions for column chromatography purification of display phages. When larger amounts of highly concentrated phage will be required, the linear gradient will be changed into a stepwise gradient where narrower peaks will be achieved and fraction collection will be easier. Based on data from the linear gradient, we will introduce conditions for the stepwise gradient for large scale purification of display phages.

### F. Immunoblot and Western Blot Analysis of Recombinant Lambda-Constructs:

To verify the expression of fusion-peptides on lambda head, immunoblot and Western blot analysis are carried out.

For immunoblot assay each phage constructs are separately plated on LB agar plate to obtain 100 to 150 plaques in each plate. The plates are incubated at 37° C. for 18 hours, until the plaques are about one mm in size. Next, a 137 mm colony/plaque screen membrane (NEN® Research products, Boston, Mass.) is soaked in distilled water and blotted dry on a filter paper. This membrane is carefully placed on the top agar and incubation was continued at 37° C. for another 15 minutes. The membrane is peeled from the agar, and washed three times with Tris saline to remove the debris and bacteria. The plates are then stored at 4° C. and the washed NEN membranes are blocked with 2% casein solution for 1 hour. After blocking, the membranes are incubated in a casein solution containing 1.25 ug/ml of diluted FB 50 monoclonal antibody. This FB50 HAAH specific monoclonal antibody was previously generated in our laboratory for diagnostic application of prostate cancer. After incubation at room temperature for two hours the membranes are washed twice in Tris saline with 0.05% Triton X-100, and once in Tris saline for 15 minutes each. The monoclonal treated membranes are incubated with 2.0 μm/ml of alkaline phosphatase labeled rabbit antimouse IgG (Kirkegaard and Perry) for one hour at room temperature. The membranes are consecutively washed three times in the same way described earlier in this procedure, followed by a final wash with 0.9% NaCl. Finally the membranes are treated with Fast Red and naphthol substrate solution for about 10 minutes and the reaction was stopped by washing the membrane in distilled water. The pink immunoreactive spots corresponds the recombinants expressing HAAH specific peptides on lambda head. For Western blots, purified lambda phage particles were electrophoresed under reducing conditions on 0.1% (w/v) SDS/10% polyacrylamide gel followed by electroblotting onto PVDF membrane (Immobilon, Millipore, Bedford, Mass.). Fusion proteins are detected either 2.5 ug/ml diluted rabbit polyclonal sera raised against recombinant expressed lambda GpD or HAAH specific E6 mouse monoclonal antibody (final concentration 1.25 ug/ml). The rabbit antisera treated membranes are incubated with 2.0 μm/ml of alkaline phosphatase labeled goat anti-rabbit IgG and mouse monoclonal treated membranes are incubated with 2.0 μm/ml of alkaline phosphatase labeled rabbit antimouse IgG for one hour at room temperature. The membranes are consecutively washed three times in the same way described earlier in plaque lift assay. Finally the membranes are treated with Fast Red and naphthol substrate solution for about 10 minutes and the reaction is stopped by washing the membrane in distilled water. immunoreactive lines correspond to the gpD specific recombinant proteins.

### Animal Experiments to Evaluate Antigenic Nature of HAAH Phage Vaccine:

### A. Study of Antigenicity of HAAH-Phage Vaccine on Female BALB/c Mice.

The purpose of this experiment is to determine the efficacy of HAAH-phage vaccine to elicit antibody response in BALB/c female mice. Previously three separate HAAH-lambda phage constructs were prepared where fragmented HAAH antigens are displayed on surface of lambda phage head as fusion of lambda capsid protein gpD. Such three constructs were designated as HAAH construct 1, HAAH construct 2, and HAAH construct 3. Four separate groups of mice (Group A, Group B, Group C, 5 mice in each group and Group D, 40 mice) will be injected subcutaneously (s/c) with various HAAH phage constructs as described in chart below (Chart 1). Briefly, group A mice will receive 5×108 pfu of HAAH construct 1 phage particles suspended in 500 μl of sterile PBS. Similarly group B and group C mice will receive same quantity of HAAH construct 2 and HAAH construct 3 phage particles respectively. Group D mice will receive equimolar mixture of all 3 phage constructs. A fifth group of mice (group E, 40 mice) will receive recombinant HAAH antigen (50 μg/mice) suspended in sterile PBS. As a control (group F, 40 mice) will be injected with wild type phage pAN-A-λ. After primary inoculation, mice will receive 1st and 2nd booster (dose will be the same as primary inoculation) of corresponding antigens at 2 weeks interval. All animals will be bled prior primary inoculation. Serum samples will be collected before every booster to monitor progression of immune response against HAAH antigens. After 21 days animal will be euthanized for final bleeding through cardiac puncture. Finally animals will be sacrificed by spinal dislocations. Sera from group D, group E and group F animals will be saved at −70° C. freezer for second animal experiment. During experiment, all animals will be monitored for their health conditions. The immune response against various HAAH-phage vaccines will be monitored by western immunoblot and ELISA.

TABLE 1

| | Groups | | | | | | |
|---|---|---|---|---|---|---|---|
| Days | A | B | C | D | E | F | Scoring |
| 0 | HAAH | HAAH | HAAH | Mixture of | Recombinant | pAN-A-λ | * |
| | construct 1 | construct 2 | construct 3 | 3 HAAH | HAAH | 5 × 10⁸ pfu | |
| | 5 × 10⁸ pfu | 5 × 10⁸ pfu | 5 × 10⁸ pfu | constructs | 50 μg | | |
| | | | | 5 × 10⁸ pfu | | | |
| 7 | HAAH | HAAH | HAAH | Mixture of | Recombinant | pAN-A-λ | |
| | construct 1 | construct 2 | construct 3 | 3 HAAH | HAAH | 5 × 10⁸ pfu | |
| | 5 × 10⁸ pfu | 5 × 10⁸ pfu | 5 × 10⁸ pfu | constructs | 50 μg | | |
| | | | | 5 × 10⁸ pfu | | | |
| 14 | HAAH | HAAH | HAAH | Mixture of | Recombinant | pAN-A-λ | |
| | construct 1 | construct 2 | construct 3 | 3 HAAH | HAAH | 5 × 10⁸ pfu | |
| | 5 × 10⁸ pfu | 5 × 10⁸ pfu | 5 × 10⁸ pfu | constructs | 50 μg | | |
| | | | | 5 × 10⁸ pfu | | | |
| 21 | Final Bleed | Final Bleed | Final Bleed | Final Bleed | Final Bleed | Final Bleed | * For 21 |
| | | | | | | | days |

Scoring: 0—normal, 1—lethargy and ruffled fur, 2—lethargy, ruffled fur and hunchback, 3—lethargy, ruffled fur, hunchback, and partially closed eyes, 4—moribund, 5—dead.

### B. Evaluation of Humoral Immunity Response Against HAAH Phage Constructs:

Previously in xenograft models of human primary liver cancer, the initial target disease, treatment with anti-HAAH antibodies reduced cancer tumor size in all animals, and in 75% of cases after four weeks of treatment tumors were kept to a non-detectable size. In a model of tumor metastasis using human colon cancer cells spreading to the liver, treatment with anti-HAAH antibodies greatly reduced the number and size of metastases. These results are highly significant and clearly indicate the utility of anti-HAAH in the treatment of human cancer. It is noteworthy that in both these instances animals were treated with antibody alone, not in conjunction with any other treatment. In this experiment, 4 groups of nude mice (Group A, Group B and Group C, and group D, 5 mice in each group) will be injected subcutaneously with a primary human liver cancer in their left flank. After 72 hours Group A, Group B and Group C nude mice will be treated by intraperitonial (i/p) route with 300 ul of sera previously collected from Group D, Group E and Group F mice of 1st animal experiment respectively. As a control Group D nude mice will be receive 300 ul of PBS. The treatment will continue every 48 hours for an additional 4 weeks. After that, the animal will be monitored for another 2 weeks without any intervention. The progression of the tumor will be monitored in treated and control groups every 48 hours to evaluate the result. Finally animals will be sacrificed by spinal dislocations and their organ will be examined by a pathologist for metastasis.

### EXAMPLE 1

### 1-HAAH-1λ Fusion Protein

### Amino Acid Sequence of HAAH-1λ Fusion Protein

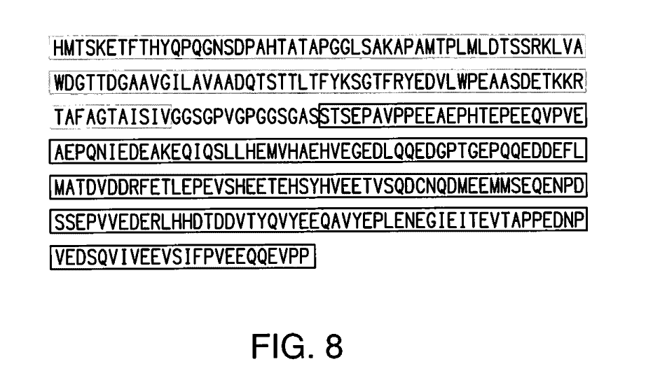

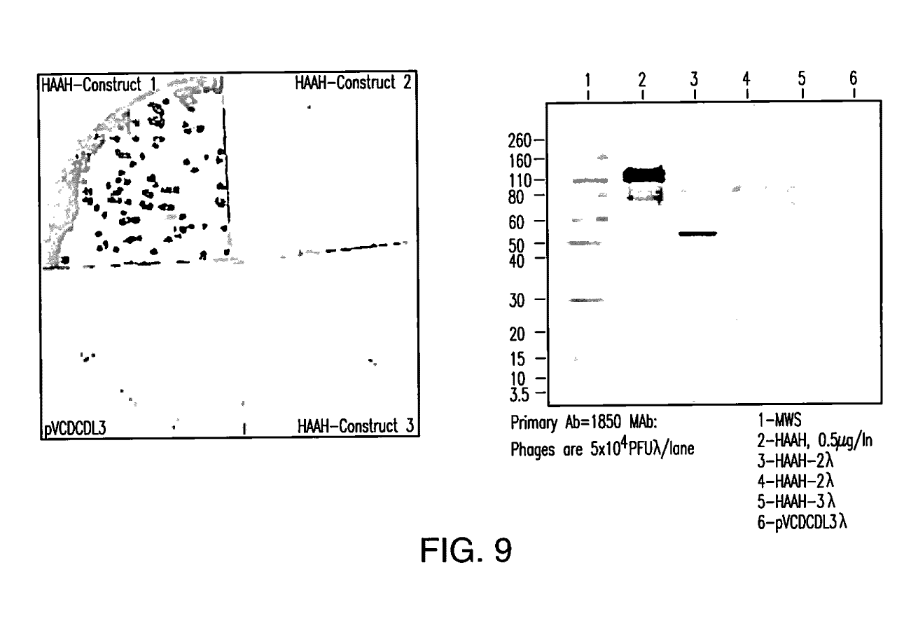

The amino acid sequence of the GpD-HAAH-1λ fusion protein is presented in FIG. 8. The total length of the open reading frame is 324 amino acids, consisting of 111 amino acids from GpD, a linker sequence and 199 amino acids (aa 113-311) of HAAH.

### Immunological Confirmation of HAAH-1λ Construct

The specific presence of the HAAH-1λ sequence was confirmed by Western blot of phage plaques and of purified phage components separated by SDS-PAGE using a murine monoclonal that binds specifically to a portion of the HAAH molecule that is contained within the HAAH-1λ construct. The antibody detects HAAH-1λ plaques, but not those of the parental phage or the other constructs. Likewise, on the blot of the SDS-PAGE gel, the antibody detects a protein of apparent molecular weight of approximately 50 kDa in HAAH-1λ (and detects recombinant full length HAAH), but exhibits no specific binding to the other constructs or parental phage. The data are presented in FIG. 9.

### Summary of Manufacturing of a Drug Substance Comprising HAAH-1λ

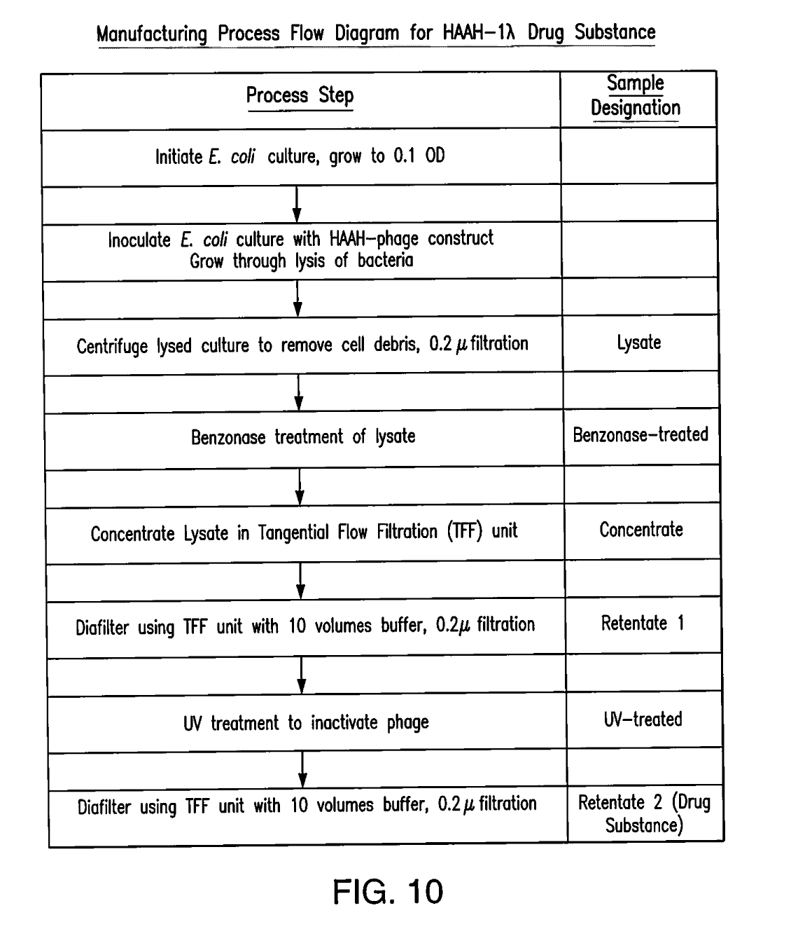

The HAAH Nanoparticle Vaccine, HAAH-1λ drug substance is prepared by growing the bacteriophage construct in E. coli. The main purification steps make use of the macromolecular size of the bacteriophage to diafilter away smaller molecular size bacterial and media components and other non-product materials using tangential flow filtration (TFF). The manufacturing process is initiated by infecting a culture of E. coli W3110 with the Working Stock of the HAAH-1λ phage construct. The culture is grown through the lysis of the bacteria (approximately 4-5 hours), then the lysed culture is centrifuged at 11,000×g to remove bacterial cell debris. The supernatant (lysate) containing the HAAH-1λ phage is collected and filtered through a 0.2 μ filter. The lysate is concentrated at least 10-fold using a Millipore Pellicon 2 Mim TFF system equipped with a 300 kDa filter. The concentrated lysate is diafiltered in the TFF apparatus using at least 10 volumes of phosphate buffered-saline, followed by filtration using a 0.2 μ filter. The phage preparation is subjected to ultraviolet light in a flow-through system to inactivate its ability to replicate, followed by a second TFF step conducted as before. The resulting material is the nanoparticle vaccine drug substance. A flow diagram of the drug substance manufacturing process is shown in FIG. 10.

### Pharmacology and Toxicology of HAAH-1λ Fusion Protein

Nonclinical studies have focused on the immunogenicity and efficacy of the PAN-301-1 (HAAH Nanoparticle Vaccine, HAAH-1λ) in rodent models. Efficacy testing has evaluated the effect of the vaccine on solid tumors and metastases. Nonclinical toxicology studies have also been completed.

PAN-301-1 (HAAH Nanoparticle Vaccine, HAAH-1λ) Test Articles used in the studies that are presented below can be described as follows:
Development Lots (multiple lots of HAAH-1λ Bulk Drug Substance)
   Purified by development processes (generally same as current process minus the ethanol precipitation step)
   UV-treated
Toxicology Lot (HAAH-1λ Bulk Drug Substance Lot #HAAH-1λ-071015TFF30P-UV)
   Purified by current process including ethanol precipitation step
   UV-treated
   Used for formulation of toxicology test articles, Lot #PAN-301-1LO-071015, Lot #PAN-301-1MD-071015 and Lot #PAN-301-1HI-071015
GMP Lot (HAAH-1λ Bulk Drug Substance Lot #151201A)
   Purified by current process including ethanol precipitation step
   UV-treated
GMP Clinical Lot (HAAH-1λ Bulk Drug Substance Lot #151216A)
   Purified by current process including ethanol precipitation step
   UV-treated
   Used for formulation of PAN-301-1 (HAAH Nanoparticle Vaccine, HAAH-1λ) clinical lots, Lot #160311A, 160309A, 160314A

### Immunogenicity of HAAH-1λ

Immunogenicity of the PAN-301-1 (HAAH Nanoparticle Vaccine, HAAH-1λ) has been evaluated in rodents. Data are presented below for four studies as examples of the vaccine immune response.

### Study PAN-16-001: Dose Response of PAN-301-1 (HAAH Nanoparticle Vaccine, HAAH-1λ) in BALB/c Mice (Non-GLP Study)

The purpose of this study was to evaluate the immunogenicity of the clinical GMP drug product materials and the effect of vaccine dose level and number of doses on immunogenicity. Test Articles: GMP Clinical Lots, PAN-301-1 (HAAH Nanoparticle Vaccine, HAAH-1λ), Lot #160311A, 160309A, 160314A
Control Article: Phosphate-Buffered Saline

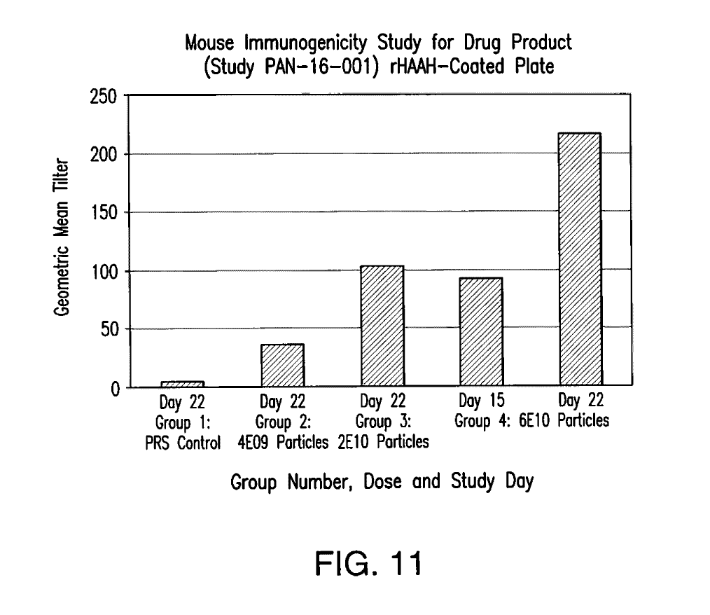

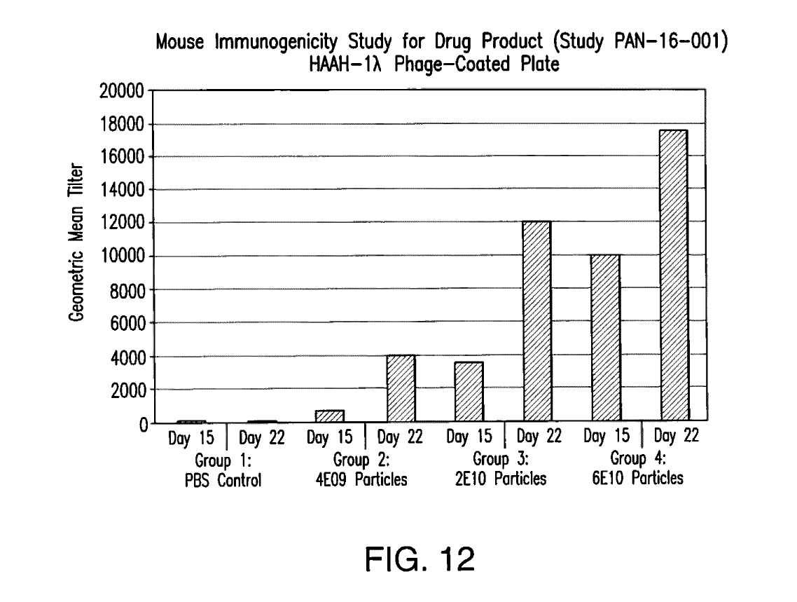

Groups of 10 BALB/c mice were immunized subcutaneously, using syringe and needle with 0.2 mL of PAN-301-1 (HAAH Nanoparticle Vaccine, HAAH-1λ) Drug Product containing 4×10⁹, 2×10¹⁰ or 6×10¹⁰ particles on Days 1, 8 and 15. Sera were obtained on Days 15 and 22. The results of antibody testing by ELISA using recombinant HAAH- or HAAH-1λ phage-coated plates are presented in FIGS. 11 and 12. Antibody to the rHAAH is specific to the HAAH target. Antibody detected on the HAAH-1λ phage is partly specific to HAAH, but also has specificity to k phage antigens. The data from both assays show a clear dose response to both the number of doses and the dose level of the vaccine.

There were no observations of local reactogenicity or adverse events associated with the administration of multiple doses of the PAN-301-1 (HAAH Nanoparticle Vaccine, HAAH-1λ).

Study TX-15-01: Dose Response of PAN-301-1 (HAAH Nanoparticle Vaccine, HAAH-1λ) in Sprague-Dawley Rats Using the 3M hMTS Intradermal Injector (GLP Study)

The purpose of this study was to evaluate the toxicity of the PAN-301-1 (HAAH Nanoparticle Vaccine, HAAH-1λ) drug product. However, as part of the repeat dose toxicity study in rats, immunogenicity of the PAN-301-1 using the 3M hMTS intradermal injector and the effect of vaccine dose level and number of doses were evaluated.

Test Articles: Toxicology Lots, PAN-301-1 (HAAH Nanoparticle Vaccine, HAAH-1λ) Lot #PAN-301-1LO-071015, Lot #PAN-301-1MD-071015 and Lot #PAN-301-1HI-071015
Control Article: Phosphate-Buffered Saline

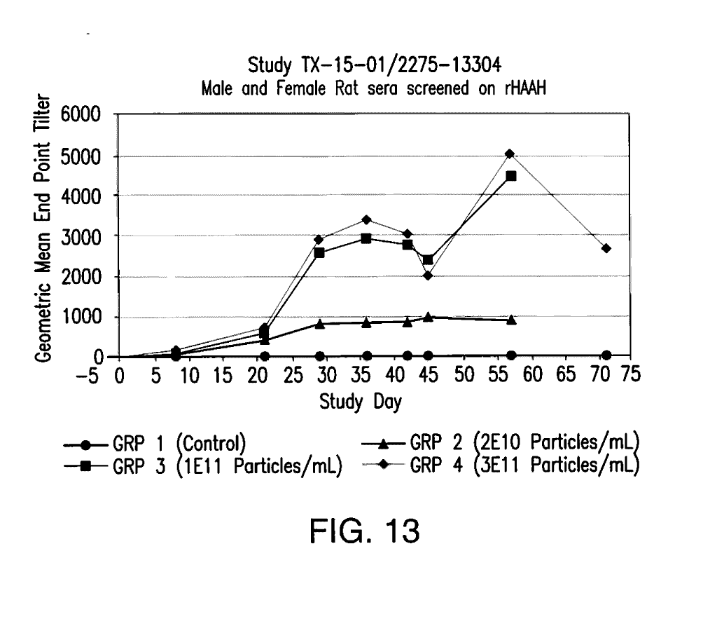

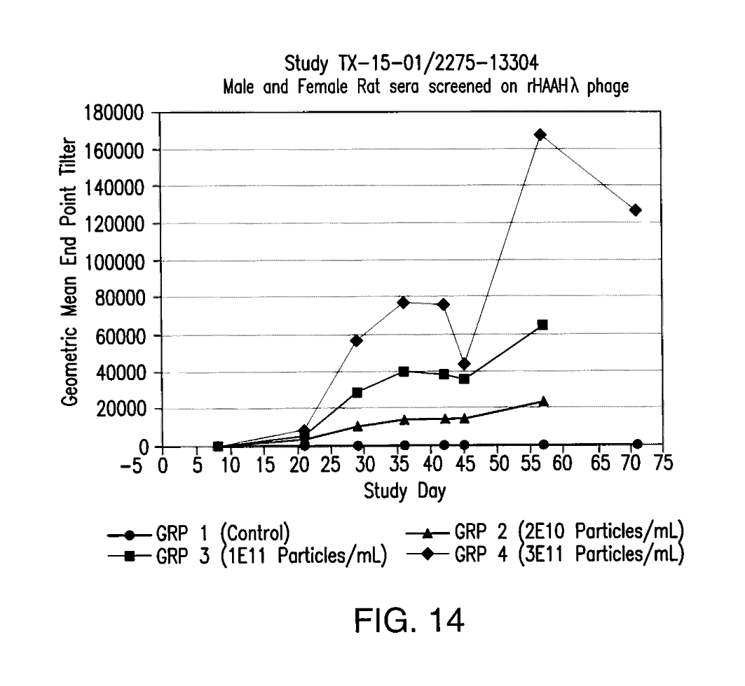

Groups of 24-36 Sprague-Dawley rats were immunized via the 3M hMTS intradermal injection device with PAN-301-1 vaccine containing 2×10¹⁰, 1×10¹¹ or 3×10¹¹ particles on Days 1, 22 and 43. Sera were obtained on multiple days throughout the study up to Day 71. The results of antibody testing by ELISA using recombinant HAAH- or HAAH-1π phage-coated plates are presented in FIGS. 13 and 14. The data from both assays show a clear dose response to both the number of doses and the dose level of the vaccine. Peak antibody response is approximately 14 days after each injection (see Days 36 and 57 in the graphs).

Generation of Cellular Immunity with HAAH-1π Vaccine in BALB/c Mice: Brown University Study (Non-GLP Study)

Cellular immunity is thought to play a significant role in the immune response to tumor cells. The ability of the PAN-301-1 (HAAH Nanoparticle, HAAH-1λ) vaccine to generate a cellular immune response was evaluated in an in vitro cytotoxic T lymphocyte (CTL) assay targeting the murine hepatoceullular carcinoma cell line BNLT3.
Test Article: Development Lot, HAAH-1λ Drug Substance, Lot #HAAH-1λ-082613CP70UV
Control Article: Phosphate-Buffered Saline

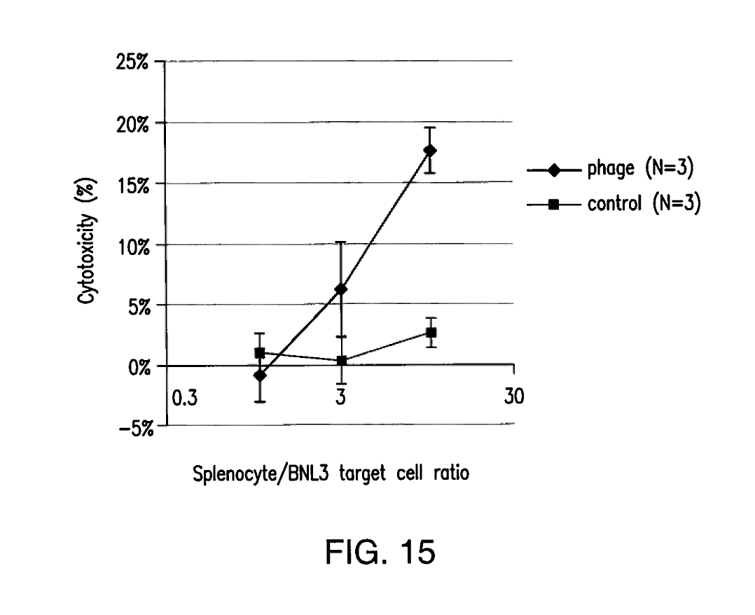

Spleen cells were obtained from BALB/c mice immunized subcutaneously using syringe and needle 4 times at weekly intervals with 2.5×10¹¹ particles of HAAH-1λ vaccine or from control mice injected with phosphate-buffered saline. The results in FIG. 15 demonstrate a robust CD8+ CTL activity.

There were no observations of local reactogenicity or adverse events associated with the administration of multiple doses of the PAN-301-1 (HAAH Nanoparticle Vaccine, HAAH-1λ).

This study was conducted in its entirety at the laboratory of Dr. J. Wands, Brown University, Providence, R.I.

### Immunogenicity of PAN-301-1 (HAAH Nanoparticle Vaccine, HAAH-1λ) Delivered Intramuscularly or Intradermally in Sprague-Dawley Rats (Non-GLP Study)

Immunogenicity of the PAN-301-1 vaccine was studied in rats, for the purpose of evaluating an intradermal injection device, the hollow Microstructured Transdermal System (hMTS) made by 3M Corporation. Since the device is designed for human use, its size precludes its use in mice, but is applicable to usage in rats. An immunogenicity study in rats was conducted to evaluate the utility of intradermal delivery of the HAAH-1λ vaccine via hMTS compared to traditional intramuscular administration with syringe and needle.
Test Article: Development Lot, HAAH-1λ Bulk Drug Substance, Lot #HAAH-1λ-101513TFFUV8HR

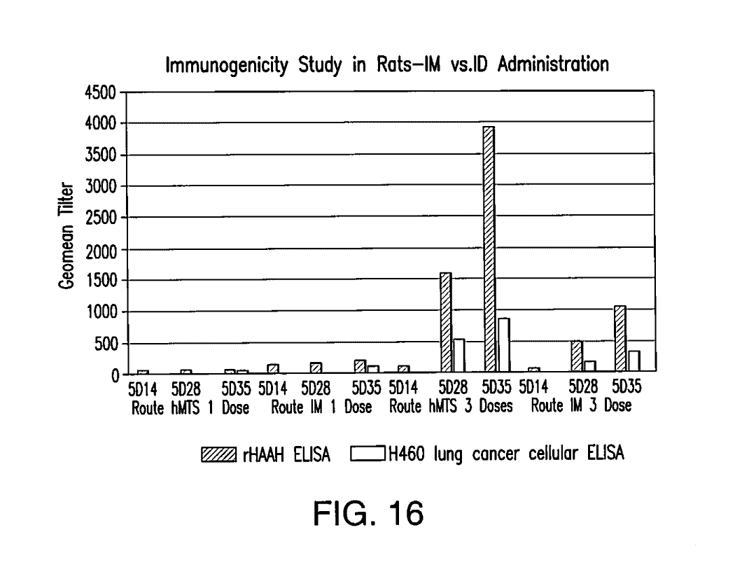

Groups of 6 rats were immunized with 2.5×10¹¹ particles (1×10¹⁰ pfu) in 0.5 mL either once at Day 1 or three times at Days 1, 14 and 28, intramuscularly via syringe and needle or intradermally using the 3M hMTS device. (An additional 16 rats were immunized 3 times with vaccine using the 3M hMTS device to gain additional data and experience). Sera were obtained at Days 14, 28 and 35 (i.e., after one, two or three injections) and were tested by recombinant HAAH ELISA or by cellular ELISA using human H460 lung cancer cells. The results are presented in FIG. 16. For rats receiving one injection, there was little difference between methods of dose administration. However, for rats receiving 3 immunizations, the intradermal administration using the 3M device gave 3-4-fold higher titers than intramuscular injection. These data demonstrate a clear advantage for the intradermal route of vaccine administration.

There were no observations of local reactogenicity or adverse events associated with the administration of multiple doses of the HAAH Nanoparticle Vaccine, HAAH-1λ.

### Efficacy of HAAH-1λ in Animals Models

The HAAH Nanoparticle Vaccines have been evaluated for efficacy in three rodent tumor cell line models of liver, breast and prostate cancers, with both solid primary tumor and metastatic outcomes.

### Mouse Hepatocellular Carcinoma Model (BNLT3 Cell Line)

BNLT3 is a BALB/c-derived hepatocellular carcinoma cell line. It produces solid tumors when administered subcutaneously and can produce metastatic tumors when injected into the spleen or peritoneum.

### Mouse Breast Cancer Model (4T1 Cell Line)

The BALB/c-derived tumor cell line, 4T1, is commonly used in mouse models of cancer. The 4T1 cell line is injected into the mammary gland, then typically forms both a solid tumor and metastasizes to other organs, such as the lung, where the number of metastatic foci may be stained and counted.

### Rat Prostate Cancer Model (MLLB-2 Cell Line)

The MLLB-2 cell line is derived from Copenhagen rats. Intracardiac/intravenous injection of the cells into rats can cause hind limb paralysis due to metastasis to the lumbar vertebrae.

### Antibodies Produced by HAAH-1λ Vaccinated Mice Bind to Tumor Cell Lines

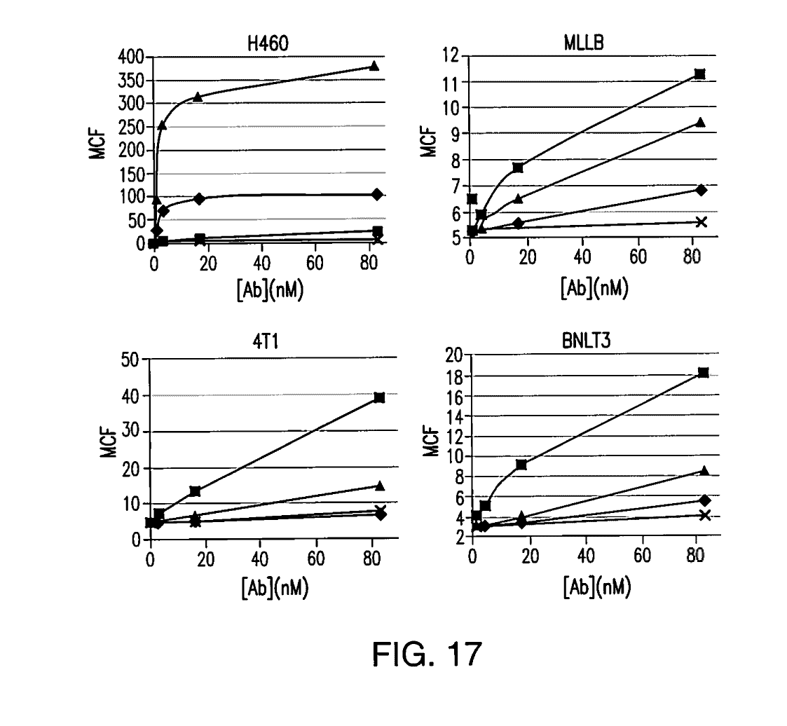

Monoclonal antibodies were isolated from HAAH-1λ-vaccinated mice by harvesting spleens, isolating and immortalizing individual B-cells and selecting those that produced antibodies reactive with HAAH. One clone in particular, 473, demonstrated particularly high binding to human tumor cells expressing HAAH. This antibody and others were used to assess the expression levels of HAAH on the surface of tumor cell lines. Anti-HAAH antibody binding was measured by flow cytometry against the human lung cancer cell line, H460, two mouse tumor lines used in in vivo challenge experiments, 4T1 and BNLT3, and one rat tumor line, also used in in vivo challenge experiments, MLLB. In FIG. 17, 473 binding (green lines) is compared to two existing murine anti-HAAH antibodies, FB50 (blue lines) and 15C7 (red lines) as well as a non-relevant control antibody (purple lines). Note the very strong binding of 473 to the human lung cancer cell line H460. Note also that at least two of the antibodies bind well to each of the tumor cell lines. These data provide verification that the 4T1, BNLT3 and MLLB-2 cell lines used in the efficacy studies presented below all express the HAAH antigen marker on their cell surfaces.

### Study PAN-13-005: Inhibition of BNLT3 Solid Tumor Growth by HAAH-1λ Immunization (Non-GLP Study)

The efficacy of the HAAH-1λ vaccine was evaluated in inhibition of growth of solid tumors upon subcutaneous injection with the mouse liver cancer cell line, BNLT3.
Test Article: Development Lot, HAAH-1λ Bulk Drug Substance, Lot #HAAH-1λ-072613CP70UV
Control Article: Parental λ Phage Lot #pVCDCDL3λ-072613CP70

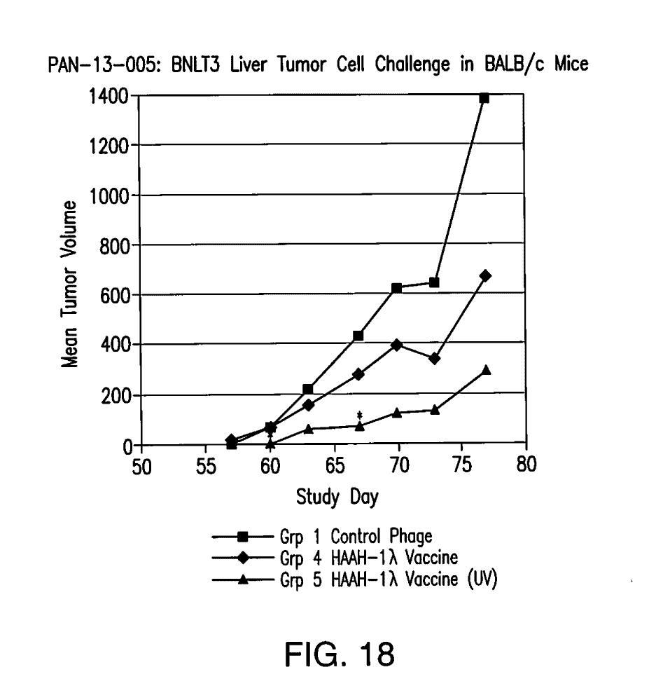

Groups of 5 BALB/c mice were immunized subcutaneously with syringe and needle on Days 1, 8, 15, 47 and 63 with 2.5×10¹¹ particles of HAAH-1λ vaccine or parental phage (λ phage that was used for producing recombinant HAAH constructs) and challenged on Day 47 with 1×10⁴ BNLT3 cells injected subcutaneously. The volume of the solid tumors was measured over 3 weeks. FIG. 18 shows an inhibition of the rate of growth of tumors in HAAH-1λ-immunized animals (UV-treated vaccine, 3 doses of vaccine) compared to the control animals and the non-UV treated HAAH-1λ parental material. Antibody levels measured in the three groups are presented in Table 2. Note that the while the antibody levels to HAAH-1λ phage are similar between the two vaccines, the UV-treated HAAH-1λ exhibits a lower titer to the rHAAH. Despite this, the inhibition of tumor growth is superior with the UV-treated vaccine.

There were no observations of local reactogenicity or adverse events associated with the administration of multiple doses of the HAAH Nanoparticle Vaccine, HAAH-1λ.

TABLE 2
Antibody Titers on HAAH-1λ-and rHAAH-Coated Plates

| | | Coating Antigen | | | |
|---|---|---|---|---|---|
| | | HAAH-1λ phage | | rHAAH | |
| Group | Dose Test Article | SD21 | SD36 | SD21 | SD36 |
| 1 | Phage λ (Lot#: | 40666.7 | 41856.0 | NT | NT |
| | pVCDCDL3λ- | | | | |
| | 072613CP70) | | | | |
| 4 | HAAH-1λ (Lot# | 79714.1 | 61677.8 | 2064.8 | 1714.8 |
| | HAAH-1λ- | | | | |
| | 072613CP70 | | | | |
| 5 | HAAH-1λ (Lot# | 101744.8 | 82225.0 | 591.8 | 604.2 |
| | HAAH-1λ- | | | | |
| | 072613CP70UV | | | | |

### Inhibition of Metastasis of BNLT3 Tumor Cells by HAAH-1λ Immunization: Brown University Study (Non-GLP Study)

The effects of HAAH-1λ vaccination on the development of metastatic BNLT3 tumors was evaluated in a study conducted at Brown University.
Test Article: Development Lot, HAAH-1λ Drug Substance, Lot #HAAH-1λ-082613CP70UV
Control Article: Phosphate-Buffered Saline

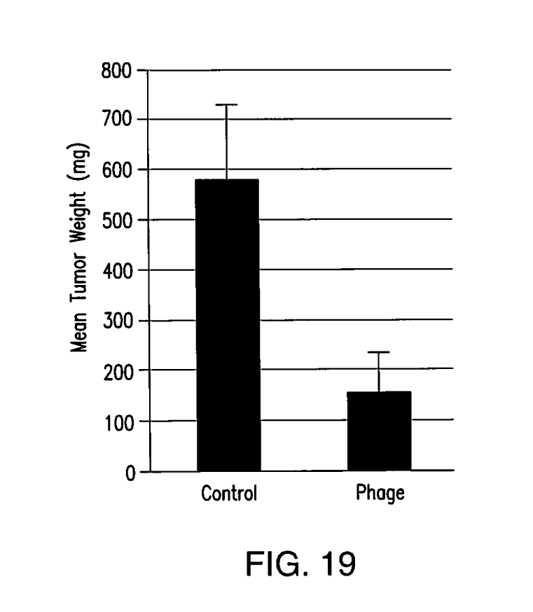

Groups of 8 mice were immunized subcutaneously 4 times at weekly intervals with 2×10¹¹ particles of HAAH-1λ vaccine or vehicle, then challenged with 1×10³ BNLT3 cells by intrasplenic injection. Mice were sacrificed to assess the weight of intra-abdominal tumors. In the control group, 7 of 8 animals had tumors, while only 3 of 8 in the vaccine group had tumors. The mean tumor weight is presented in FIG. 19. The vaccine group had a significantly lower mean tumor weight compared to the control group.

There were no observations of local reactogenicity or adverse events associated with the administration of multiple doses of the HAAH Nanoparticle Vaccine, HAAH-1λ.

This study was conducted in its entirety at the laboratory of Dr. J. Wands, Brown University, Providence, R.I.

### Study 15-005: Inhibition of 4T1 Solid Tumor Growth by HAAH-1λ Immunization (Non-GLP Study)

The efficacy of the HAAH-1λ vaccine was evaluated in inhibition of growth of solid tumors upon subcutaneous injection with the 4T1 mouse breast cancer cell line.
Test Articles: Development Lot, HAAH-1λ Bulk Drug Substance, Lot # HAAH-1λ-020415TFFUV30M
   Toxicology Lot, HAAH-1λ Bulk Drug Substance, Lot #HAAH-1λ-071015TFF30P-UV
   GMP Lot, HAAH-1λ Bulk Drug Substance, Lot #151201A
Control Article: Parental λ Phage Lot #pVCDCDL3λ-091415TFF30P-UV

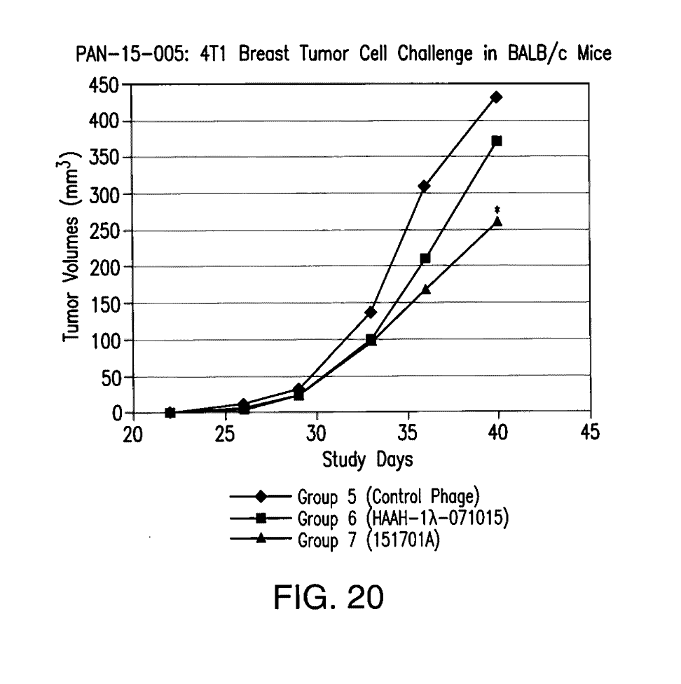

In a study that measured solid mammary gland tumors, groups of 8 BALB/c mice were immunized subcutaneously with 3×10¹¹ particles of HAAH-1λ vaccine or parental lambda phage as control on Days 1, 8, 15 and 29 using a syringe and needle. A challenge dose of 2×10⁴ 4T1 cells was injected into the mammary gland on Day 20 for one control group and two vaccine groups. The solid tumors were measured over a 3 week period. The data are presented in FIG. 20. Both vaccine groups had lower tumor growth than the control group and the GMP Lot #151201A had significantly lower mean tumor volume at Day 40.

Antibody levels are comparable among vaccine lots and are presented in Table 3.

There were no observations of local reactogenicity or adverse events associated with the administration of multiple doses of the PAN-301-1 (HAAH Nanoparticle Vaccine, HAAH-1λ).

TABLE 3
Antibody Levels on HAAH-1λ-and rHAAH-Coated Plates

| | | Coating Antigen | | | | | |
|---|---|---|---|---|---|---|---|
| | | HAAH-1λ phage | | | rHAAH | | |
| Grp | Dose Test Articles | SD22 | SD36 | SD43 | SD22 | SD36 | SD43 |
| 5 | Phage λ, Lot: | 21611.5 | 34256.6 | 39221.7 | <50 | <50 | <50 |
| | pVCDCDL3λ- | | | | | | |
| | 091415TFF30P-UV | | | | | | |
| 6 | (HAAH-1λ, Lot: | 22503.0 | 30693.0 | 32526.3 | 132.8 | 398.0 | 460.3 |
| | HAAH-1λ- | | | | | | |
| | 071015TFF30P-UV | | | | | | |
| 7 | HAAH-1λ, Lot: | 22752.1 | 33204.7 | 34054.1 | 93.3 | 368.2 | 461.9 |
| | HAAH-1λ-151201A | | | | | | |

### Study PAN-15-005: Inhibition of Metastasis of 4T1 Breast Tumor Cells by HAAH-1λ Immunization (Non-GLP Study)

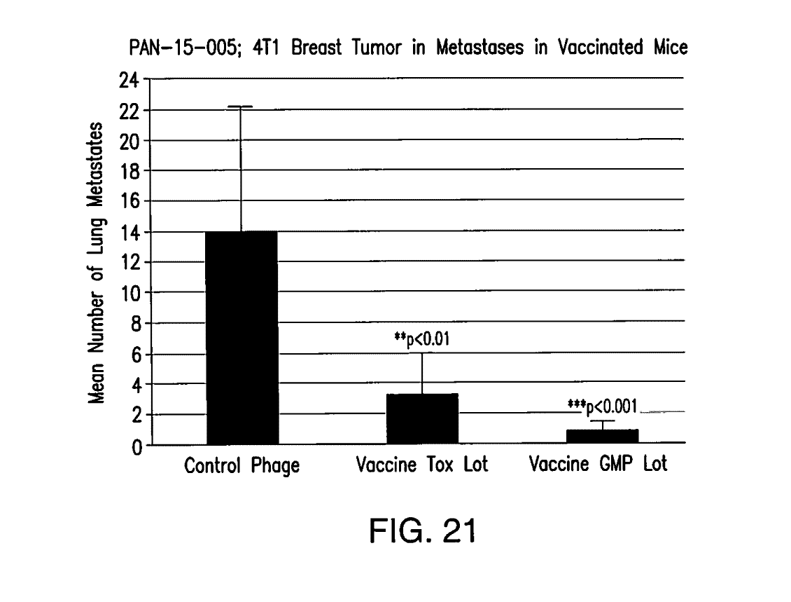

In addition to the evaluation of the effect of HAAH-1λ vaccine on 4T1 solid tumors, the effect of the vaccine on metastasis of 4T1 cells to the lungs was assessed on the same animals as presented in the section above for the solid tumors. Upon sacrifice at Day 41, the lungs of these same groups of mice were dissected, stained and the metastases were counted. The data are presented in FIG. 21. Both lots of the HAAH-1λ Bulk Drug Substance lowered the number of lung metastases highly significantly compared to the control group. These data are consistent with the proposed intended utility of the vaccine in reducing metastases or recurrence of cancer.

There were no observations of local reactogenicity or adverse events associated with the administration of multiple doses of the HAAH Nanoparticle Vaccine, HAAH-1λ.

### Studies PAN-14-004, PAN-15-004, PAN-15-005: Inhibition of Metastasis of 4T1 Breast Tumor Cells by HAAH-1λ Immunization (Non-GLP Studies)

The 4T1 lung metastasis study was conducted in a similar manner 3 times with similar positive results for the HAAH-1λ vaccine (presented in Table 4). The following materials were used for Study PAN-14-004:
Test Article: Development Lot, HAAH-1λ Bulk Drug Substance, Lot #HAAH-1λ-101513TFFUV
Control Article: Parental λ Phage Lot #pVCDCDL3λ-072613CP70

Groups of 8 BALB/c mice were immunized subcutaneously with 2.5×10¹¹ particles of HAAH-1λ vaccine or parental lambda phage as control on Days 1, 8 and 22 using a syringe and needle. A challenge dose of 2×10⁴ 4T1 cells was injected into the mammary gland on Day 15 for one control group and two HAAH-1λ vaccine groups and a challenge dose of 7.5×10³ 4T1 cells was injected into the mammary gland on Day 15 for a second set of one control group and two HAAH-1λ vaccine groups. The animals were sacrificed at Day 43 for dissection of lungs for metastases counts.

The following materials were used for Study PAN-15-004:
Test Articles: Development Lot, HAAH-1λ Bulk Drug Substance, Lot #HAAH-1λ-111414TFFUV2HR
   Toxicology Lot, HAAH-1λ Bulk Drug Substance, Lot #HAAH-1λ-071015TFF30P-UV
Control Article: Parental λ Phage Lot #pVCDCDL3λ-072613CP70

Groups of 8 BALB/c mice were immunized subcutaneously with 2×10¹¹ particles of HAAH-1λ vaccine or parental lambda phage as control on Days 1, 8, 22, 29 and 36 using a syringe and needle. A challenge dose of 2×10⁴ 4T1 cells was injected into the mammary gland on Day 15 for one control group and two HAAH-1λ vaccine groups. The animals were sacrificed at Day 43 for dissection of lungs for metastases counts.

Table 4 presents the metastases data for the two studies described above, plus PAN-15-005 that was described in the previous sections. Note that for all three studies, the challenge with 2×10⁴ 4T1 tumor cells showed results of greatly reduced number of metastases in the vaccine compared to the control groups, even in the low dose vaccine group (2.5×10¹⁰ particles) of PAN-14-004. The differences were significant or highly significant for Studies PAN-14-004 and PAN-15-005. In Study PAN-15-004, the control group animals had variable numbers of metastases (0-74 metastases/mouse) such that the statistical test of difference did not show significance. Curiously, only one pairwise comparison, the low challenge dose in Study PAN-14-004 did not show reduction in metastases in the vaccine group. These data provide strong support that the PAN-301-1 (HAAH Nanoparticle Vaccine, HAAH-1λ) can inhibit metastasis of tumors.

TABLE 4
4T1 Lung Metastases Data for 3 Studies in BALB/c Mice

| | | | | Mean | Mean |
|---|---|---|---|---|---|
| | # Cells | Dose Vaccine or | Necropsy | Control | Vaccine |
| Study # | Injected/Day | Control/Days | Day | Metastases | Metastases |
| PAN-14-004 | 2 × 10⁴/Day 15 | 2.5 × 10¹¹/Day 1, 8, 22 | Day 43 | 47.9 | 27.8* |
| | | 2.5 × 10¹⁰/Day 1, 8, 22 | | | 11.0*** |
| | 7.5 × 10³/Day | 2.5 × 10¹¹/Day 1, 8, 22 | | 30.1 | 39.9 |
| PAN-15-004 | 2 × 10⁴/Day 15 | 2 × 10¹¹/ | Day 43 | 17.4 | 8.0 |
| | | Day 1, 8, 22, 29, 36 | | | 2.8 |
| PAN-15-005 | 2 × 10⁴/Day 20 | 3 × 10¹¹/ | Day 41 | 14.0 | 3.2** |
| | | Day 1, 8, 15, 29 | | | 0.7*** |

*p < 0.05,
**p < 0.01,
***p < 0.001

Study PAN-14-003: Increased Survival of Mice 4T1 Tumors by Immunization with HAAH-1λ (Non-GLP)

One study with the 4T1 cell line had unexpected results which are described below. The following materials were used for Study PAN-14-003:
Test Article: Development Lot, HAAH-1λ Bulk Drug Substance, Lot #HAAH-1λ-101513TFFUV
Control Article: Parental λ Phage Lot #pVCDCDL3λ-072613CP70

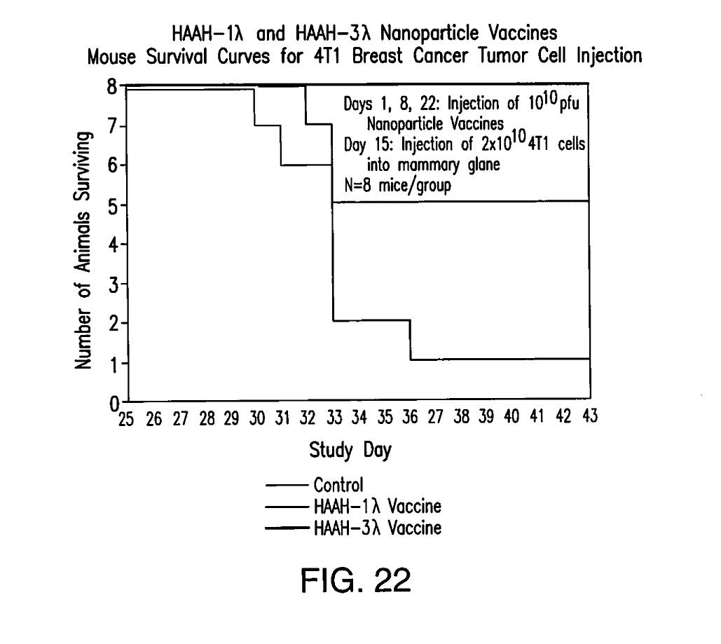

Groups of 8 BALB/c mice were immunized subcutaneously using a syringe and needle with 2.5×10¹¹ particles of the HAAH-1λ vaccine, another construct, HAAH-3λ, and control phage on Days 1, 8 and 22 and challenged with 2×10⁴ 4T1 cells on Day 15. The challenge dose of 4T1 cells in this study was particularly aggressive, resulting in high levels of mortality (7 of 8 animals) in the control group. Interestingly, there were only 3 of 8 animals dead in the HAAH-1λ group and none in the HAAH-3λ group. The data are presented in FIG. 22.

There were no observations of local reactogenicity or adverse events associated with the administration of multiple doses of the HAAH Nanoparticle Vaccine, HAAH-1λ.

The animal portion of this study was conducted for Panacea Pharmaceuticals by Smithers Avanza, Gaithersburg, Md. There were no samples for antibody testing due to the high mortality.

### Study PAN-15-001: Rat Prostate Cancer Model (MLLB-2 Cell Line)(Non-GLP Study)

A rat model of prostate cancer metastasis was evaluated. The MLLB-2 cell line is derived from Copenhagen rats and can cause hind limb paralysis due to metastasis to the lumbar vertebrae. The study protocol and study report for PAN-15-001 are presented in Appendix 8.10. The study materials for PAN-15-001 were:
Test Article: Development Lot, HAAH-1λ Bulk Drug Substance, Lot # HAAH-1λ-101513TFFUV
Control Article: No treatment

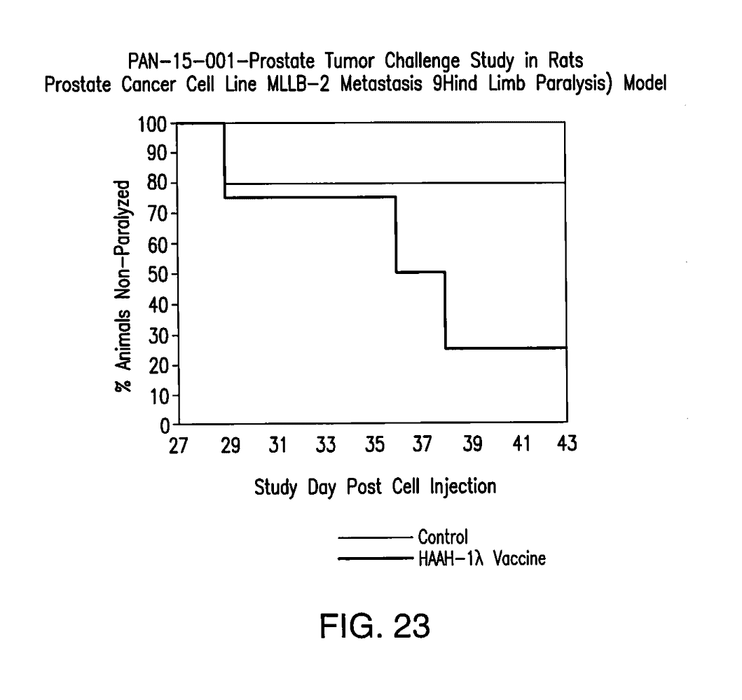

In a pilot study, five Copenhagen rats were given intradermal injections of 2.5×10¹¹ particles of HAAH-1λ on Days 1, 15 and 29 using the 3M hMTS injection device, while four animals were untreated. MLLB-2 cells were injected intra-arterially on Day 1 (7.5×10³−2×10⁴ cells). Rats were observed daily for hind limb paralysis. In this study, 3 of 4 control rats had hind limb paralysis compared to only 1 of 5 vaccinated animals (data shown in FIG. 23).

There were no observations of local reactogenicity or adverse events associated with the administration of multiple doses of the HAAH Nanoparticle Vaccine, HAAH-1λ.

### Safety Observations

The HAAH-1λ vaccine has been used to evaluate immunogenicity and efficacy in three independent laboratories. In all studies at each laboratory, the vaccine has exhibited excellent tolerability and safety. It is important to note that there have been no local reactogenicity or adverse events associated with the administration of multiple doses of the HAAH Nanoparticle Vaccine in mice and rats. These vaccines are immunogenic, show efficacy in 3 tumor model systems and are safe in the doses given to rodents.

### SUMMARY

The HAAH-1λ vaccine has been evaluated in rodents for immunogenicity and efficacy. The nonclinical data generated to date include:
Demonstration of immunogenicity in mice and rats
Demonstration of dose response to both amount of vaccine and number of doses of vaccine
Demonstration of both humoral and cellular immunity
Demonstration of enhanced immunogenicity for intradermal compared to intramuscular immunization
Efficacy data in three rodent tumor models, including solid tumors and metastases
   Inhibition of solid tumor growth and metastases with the mouse hepatocellular carcinoma cell line BNLT3
   Inhibition of solid tumor growth, lung metastases and mortality with the mouse breast cancer cell line 4T1
   Inhibition of metastases with the rat prostate cancer cell line MLLB-2

The immunogenicity and efficacy data presented above have been generated in three independent laboratories which provides corroboration of the nonclinical observations in multiple environments and with multiple technical staff. It is important to note that there have been no local reactogenicity or adverse events associated with the administration of multiple doses of the HAAH Nanoparticle Vaccine in mice and rats. These vaccines are immunogenic, show efficacy in 3 tumor model systems and are safe in the doses given to rodents.

### Description of Toxicology Study Design

Panacea Pharmaceuticals has conducted a single repeated dose toxicity study in rats that has been designed to provide toxicology data to support the planned multiple dose Phase 1 study of the PAN-301-1 (HAAH Nanoparticle Vaccine, HAAH-1λ) in humans.
Study site: Smithers Avanza, Gaithersburg, Md. in place of MPI Reseach, Mattawan Mich.
Sprague Dawley rats were used in place of the CD rat.
The identical clinical product and formulations were used in this rat study. The three dose levels for the study were 2×10¹⁰, 1×10¹¹ and 3×10¹¹ particles formulated in a 1 mL volume in phosphate-buffered saline and filled into the 3M glass cartridge with bromobutyl rubber stopper and crimp cap.
The study included assessment to verify that the pre-specified dose level of PAN-301-1 was delivered to the rats
The humoral immune response to HAAH-1λ phage and HAAH was determined. Additional time points (increased to 8 time points after the first dose of vaccine) were added to capture the peak and subsequent decline of response.
The dosing units were based on particles.
An extended recovery period sacrifice was added at Day 71 for the control and high dose groups to capture the post-antibody-peak recovery parameters.

The study was conducted in compliance with GLP according to the following design:
Study Title: PAN-301-1: A Seven-Week Intradermal Toxicity Study in Sprague-Dawley Rats with Two-Week and Four-Week Recovery Periods
Study Number: Panacea Number: TX-15-01, Smithers Avanza Number: 2275-13304
Testing Facility Smithers Avanza Toxicology Services
   11 Firstfield Road
   Gaithersburg, Md. 20878-1704
Animals: 120 (60/sex) Sprague-Dawley rats, obtained from ENVIGO, Frederick Md.
Test Article: PAN-301-1 (HAAH Nanoparticle Vaccine, HAAH-1λ), tested at three dose levels. The PAN-301-1 doses were prepared from HAAH-1λ Bulk Drug Substance Lot #HAAH-1λ-071015TFF-30P-UV. Certificates of Analysis for the Drug Substance and the 3 PAN-301-1 test articles are presented in the Audited Study Report in Appendix 8.1
Control Article: Phosphate-Buffered Saline (same material as Test Article diluent).
Dosing: Intradermal injection using the 3M hMTS device on Days 1, 22 and 43. Three dose levels of vaccine are used per Table 8.4.
Sacrifice and Necropsy: Main study and recovery phase sacrifices and necropsies on Days 45, 57 and 71 per Table 5.
Parameters Evaluated: Table 6 below.

TABLE 5
Study Design for Study TX-15-01

| | | Nominal | Nominal | Number of Animals | | | | | |
|---|---|---|---|---|---|---|---|---|---|
| | | Test Article | Test Article | | | 14-Day | | 28-Day | |
| | | Dosage | Concentration | Main Phase | | Recovery Phase | | Recovery Phase | |
| Group | Treatment | (particles) | (particles/mL) | Males | Females | Males | Females | Males | Females |
| 1 | PBS | 0 | 0 | 6 | 6 | 6 | 6 | 6 | 6 |
| 2 | PAN-301-1 | 2 × 10¹⁰ | 2 × 10¹⁰ | 6 | 6 | 6 | 6 | — | — |
| 3 | PAN-301-1 | 1 × 10¹¹ | 1 × 10¹¹ | 6 | 6 | 6 | 6 | — | — |
| 4 | PAN-301-1 | 3 × 10¹¹ | 3 × 10¹¹ | 7 | 6 | 6 | 6 | 5 | 6 |

PBS—Sterile phosphate bufferedsaline

TABLE 6
Parameters Evaluated for Study TX-15-01
Parameter

Mortality
Physical Examinations
Cageside Examinations
Draize Observations
Body Weights
Body Weight Changes
Food Consumption
Body Temperatures
Ophthalmologic Examinations
Clinica Pathology (clinical chemistry, hematology, coagulation)
Gross Pathology
Absolute and Relative Organ Weights
Histopathology Findings
Antibody Levels to HAAH and HAAH-1λ phage

The significant study dates are as follows:

| | |
|---|---|
| Study initiation date | 13 Oct. 2015 |
| Receipt of Animals | 13 Oct. 2015 |
| First day of dosing (males) | 19 Oct. 2015 |
| First day of dosing (females) | 20 Oct. 2015 |
| Last day of dosing (males) | 30 Nov. 2015 |
| Last day of dosing (females) | 1 Dec. 2015 |
| Main study necropsy (males) | 2 Dec. 2015 |
| Main study necropsy (females) | 3 Dec. 2015 |
| Recovery 1 (males) | 14 Dec. 2015 |
| Recovery 1 (females) | 15 Dec. 2015 |
| Recovery 2 (males) | 28 Dec. 2015 |
| Recovery 2 (females) | 29 Dec. 2015 |

The efficiency of the 3M hMTS device to deliver the pre-specified dose volume (1 mL) to the animals was evaluated in the course of the 3 dosings for each animal in the following manner. A skin flap of the dorsal thoracic region was pulled over a block fixture to tauten the skin and then the skin was taped in place using 3M Durapore™ medical tape. The adhesive liner was removed and the hMTS injector was applied to the marked area of skin. After waiting at least 10 seconds (to aid skin adhesion), the microneedles of the hMTS injector were inserted into the skin and the test or the control article were pressurized which initiated the injection. When the plunger reached the end of travel the injection was complete, and the dose time was recorded.

The hMTS injector was left on the skin for at least two minutes after the injection was complete to allow for depressurization. The hMTS injector was removed by peeling it off the skin, the injection site was blotted with gauze and the gauze was weighed to determine the amount of dose that was not administered intradermally (termed the residual dose weight). The summary results of this testing are presented in Table 8.6 (determinations for each animal are presented in the Audited Draft Report in Appendix 8.1). Nearly half (177) of the 360 doses were fully injected into the rats and an additional 40% had less than 0.1 mL residual dose (generally much less than 0.1 mL) not injected. These data support efficiency and relevance of the hMTS dosing in the rats. Additionally, for those animals that received ≦50% of the dose on SD 1 or ≦70% at subsequent dosing intervals, the same site was dosed again with the same test or control article.

TABLE 7
Summary of Residual Dose Weights

| | | # Animals at each Residual Dose Weight | | | | |
|---|---|---|---|---|---|---|
| | | | | 0.1 < | 0.3 < | |
| Group/ | Dose | No | <0.1 | X < 0.3 | X < 0.6 | Re- |
| Sex | # | Residual | g (mL) | g (mL) | g (mL) | dose |
| Group | 1 | 7 | 8 | 0 | 2 | 1 |
| 1Male | 2 | 4 | 11 | 2 | 0 | 1 |
| | 3 | 9 | 8 | 0 | 0 | 1 |
| Group | 1 | 8 | 4 | 0 | 0 | 0 |
| 2 Male | 2 | 4 | 8 | 0 | 0 | 0 |
| | 3 | 10 | 7 | 0 | 0 | 0 |
| Group | 1 | 3 | 6 | 2 | 0 | 1 |
| 3 Male | 2 | 5 | 7 | 0 | 0 | 0 |
| | 3 | 5 | 7 | 0 | 0 | 0 |
| Group | 1 | 8 | 8 | 1 | 0 | 1 |
| 4 Male | 2 | 4 | 11 | 0 | 0 | 3 |
| | 3 | 15 | 3 | 0 | 0 | 0 |
| Group | 1 | 6 | 9 | 2 | 0 | 1 |
| 1 Female | 2 | 7 | 6 | 3 | 0 | 2 |
| | 3 | 13 | 4 | 0 | 0 | 1 |
| Group | 1 | 4 | 6 | 0 | 0 | 2 |
| 2 Female | 2 | 5 | 5 | 2 | 0 | 0 |
| | 3 | 7 | 5 | 0 | 0 | 0 |
| Group | 1 | 8 | 2 | 1 | 0 | 0 |
| 3 Female | 2 | 6 | 5 | 1 | 0 | 0 |
| | 3 | 9 | 3 | 0 | 0 | 0 |
| Group | 1 | 9 | 5 | 3 | 0 | 1 |
| 4 Female | 2 | 12 | 3 | 2 | 0 | 1 |
| | 3 | 7 | 6 | 3 | 0 | 2 |

The feasibility of performing intradermal injections in the rats using the hMTS device was evaluated and confirmed by 3M. The hMTS device delivers drug at a level ˜800 μm deep, about ½ to ⅓ the thickness of rat and human skin.

### Results of the Toxicology Study

Levels of antibody to both recombinant HAAH and to the recombinant bacteriophage construct HAAH-1λ were within background in control animals. However, all three groups vaccinated with PAN-301-1 showed readily measurable antibody to both recombinant HAAH and to the recombinant bacteriophage construct HAAH-1λ. These groups demonstrated a dose- and dose number-dependent specific antibody response to the HAAH target when tested by ELISA. Tables 8 and 9 provide the group geometric means for the testing time points for rHAAH-coated plates and HAAH-1λ phage-coated plates, respectively. For each antigen, the peak antibody titer is generally reached at 14 days after the previous immunization, i.e., Day 36 and Day 57, followed by a decline at the next time point. For this series of three injections, the peak titer was successively higher after each immunization.

TABLE 8
Geometric mean end-point titer of TX-15-01 rat sera on rHAAH-coated plates

| | Study Day | | | | | | | | |
|---|---|---|---|---|---|---|---|---|---|
| | −3 | 8 | 21 | 29 | 36 | 42 | 45 | 57 | 71 |
| Group 1 | N = 8 | N = 36 | N = 36 | N = 36 | N = 36 | N = 29 | N = 12 | N = 12 | N = 12 |
| | <20 | 27 | 27 | 26 | 25 | 27 | 20 | 29 | 27 |
| Group 2 | N = 8 | N = 24 | N = 24 | N = 24 | N = 24 | N = 24 | N = 12 | N = 12 | N/A |
| | <20 | 109 | 403 | 786 | 893 | 836 | 962 | 872 | N/A |
| Group 3 | N = 8 | N = 24 | N = 24 | N = 24 | N = 24 | N = 24 | N = 12 | N = 12 | N/A |
| | <20 | 85 | 574 | 2532 | 2887 | 2768 | 2380 | 4465 | N/A |
| Group 4 | N = 8 | N = 36 | N = 36 | N = 36 | N = 36 | N = 36 | N = 12 | N = 12 | N = 11 |
| | <20 | 179 | 724 | 2875 | 3379 | 2987 | 1988 | 4973 | 2666 |

TABLE 9
Geometric mean end-point titer of TX-15-01 rat sera on HAAH-1λ phage-coated plates

| | Study Day | | | | | | | | |
|---|---|---|---|---|---|---|---|---|---|
| | −3 | 8 | 21 | 29 | 36 | 42 | 45 | 57 | 71 |
| Group 1 | N = 8 | N = 36 | N = 36 | N = 36 | N = 36 | N = 29 | N = 12 | N = 12 | N = 12 |
| | 22 | 23 | 25 | 28 | 31 | 33 | 23 | 52 | 131 |
| Group 2 | N = 8 | N = 24 | N = 24 | N = 24 | N = 24 | N = 24 | N = 12 | N = 12 | N/A |
| | 39 | 146 | 3216 | 10689 | 14278 | 14004 | 13935 | 23659 | N/A |
| Group 3 | N = 8 | N = 24 | N = 24 | N = 24 | N = 24 | N = 24 | N = 12 | N = 12 | N/A |
| | 22 | 197 | 5280 | 28891 | 38884 | 37975 | 35479 | 64917 | N/A |
| Group 4 | N = 8 | N = 36 | N = 36 | N = 36 | N = 36 | N = 36 | N = 12 | N = 12 | N = 11 |
| | 34 | 466 | 8980 | 56878 | 76837 | 75104 | 43190 | 165938 | 126037 |

Treatment with PAN-301-1 at nominal doses up to 3×10¹¹ particles had no effect on mortality, physical examinations, cage side observations, dermal Draize observations, body weights or body weight changes, food consumption, body temperature, ophthalmologic observations, gross pathology, absolute and relative organ weights, hematology, or clinical chemistry.

At the terminal necropsy (SD 45), mean prothrombin time (PT) was slightly prolonged in males given ≧1×10¹¹ particles and females given 3×10¹¹ particles. This alteration was considered non-adverse. Test article-related microscopic findings were present at the injection site in animals given ≧1×10¹¹ particles, and consisted of mild or moderate mononuclear or mixed inflammatory cell infiltrates in the dermis and/or subcutis. These findings were considered non-adverse.

At the first recovery necropsy (SD 57), after a 2-week recovery period, mean PT was slightly prolonged in males given ≧1×10¹¹ particles and females given 3×10¹¹ particles. The difference from controls was similar to SD 45.

At the second recovery necropsy (SD 71), after a 4-week recovery period, the mean PT was minimally prolonged in females given 3×10¹¹ particles. The difference from controls was less than at earlier time points, and this alteration was not present in males, which suggests ongoing recovery. There were fewer microscopic findings at the injection site suggesting resolution of observations noted in animals at the terminal necropsy.

In conclusion, treatment with PAN-301-1 at nominal doses up to 3×10¹¹ particles had no effect on mortality, physical examinations, cage side observations, dermal draize observations, body weights or body weight changes, food consumption, body temperature, ophthalmologic observations, gross pathology, absolute and relative organ weights, hematology, or clinical chemistry. A slightly prolonged but non-adverse PT time in males given ≧1×10¹¹ particles and females given 3×10¹¹particles persisted through the first recovery interval. The prolonged PT time resolved in males by the second recovery interval but remained minimally prolonged in females given 3×10¹¹ particles. Test article-related microscopic findings were present at the injection site in animals given ≧1×10¹¹ particles, and consisted of mild or moderate mononuclear or mixed inflammatory cell infiltrates in the dermis and/or subcutis. These findings were considered non-adverse and resolved during recovery.

While the invention has been described with reference to certain exemplary embodiments thereof, those skilled in the art may make various modifications to the described embodiments of the invention without departing from the scope of the invention. The terms and descriptions used herein are set forth by way of illustration only and not meant as limitations. In particular, although the present invention has been described by way of examples, a variety of compositions and processes would practice the inventive concepts described herein. Although the invention has been described and disclosed in various terms and certain embodiments, the scope of the invention is not intended to be, nor should it be deemed to be, limited thereby and such other modifications or embodiments as may be suggested by the teachings herein are particularly reserved, especially as they fall within the breadth and scope of the claims here appended. Those skilled in the art will recognize that these and other variations are possible within the scope of the invention as defined in the following claims and their equivalents.

What is claimed it:

1. A bacteriophage comprising at least one amino acid sequence native to Aspartyl-[Asparaginyl]-.beta.-hydroxylase.

2. The bacteriophage of claim 1, wherein the at least one amino acid sequence native to Aspartyl-[Asparaginyl]-.beta.-hydroxylase is selected from the group consisting of the amino acid sequence of Construct I.

3. The bacteriophage of claim 1, wherein the bacteriophage comprises the amino acid sequence of Construct II.

4. The bacteriophage of claim 1, wherein the bacteriophage comprises the amino acid sequence of Construct III.

5. The bacteriophage of claim 1, wherein the bacteriophage is selected from the group consisting of Lambda, T4, T7, and M13/f1.

6. The bacteriophage of claim 5, wherein the bacteriophage is bacteriophage Lambda.

7. A method for treating cancer comprising the step of providing a patient with an immune system stimulating amount of the bacteriophage of claim 1.

8. A nucleic acid construct comprising at least one nucleotide sequence encoding an amino acid sequence native to Aspartyl-[Asparaginyl]-.beta.-hydroxylase and a nucleotide sequence encoding gpD.

9. The nucleic acid construct of claim 8, wherein the at least one amino acid sequence native to Aspartyl-[Asparaginyl]-.beta.-hydroxylase is the amino acid sequence of Construct I.

10. The nucleic acid construct of claim 8, wherein the at least one amino acid sequence native to Aspartyl-[Asparaginyl]-.beta.-hydroxylase is the amino acid sequence of Construct II.

11. The nucleic acid construct of claim 8, wherein the at least one amino acid sequence native to Aspartyl-[Asparaginyl]-.beta.-hydroxylase is the amino acid sequence of Construct III.

12. A recombinant Lambda phage comprising the nucleic acid construct of claim 8.

13. A composition comprising nano-particles, wherein the nano-particles further comprise at least one amino acid sequence native to Aspartyl-[Asparaginyl]-.beta.-hydroxylase.

14. The composition of claim 13, wherein the at least one amino acid sequence native to Aspartyl-[Asparaginyl]-.beta.-hydroxylase is the amino acid sequence of Construct I.

15. The composition of claim 13, wherein the nano-particle comprises the amino acid sequence of Construct II.

16. The composition of claim 13, wherein the nano-particle comprises the amino acid sequence of Construct III.

17. A method for treating cancer comprising the step of providing a patient with an immune system stimulating amount of the composition of claim 13.

18. A method for treating cancer comprising the step of contacting dendritic cells of a patient with an immune system stimulating amount of the composition of claim 13.

19. A method for treating cancer comprising the step of providing an immune system stimulating amount of Lambda phage to a patient, wherein the Lambda phage comprise amino acid sequences native to Aspartyl-[Asparaginyl]-.beta.-hydroxylase expressed on their surface.

20. The method of claim 19, wherein the amino acid sequences native to Aspartyl-[Asparaginyl]-.beta.-hydroxylase comprise the amino acid sequence of Construct I, the amino acid sequence of Construct II and the amino acid sequence of Construct III.


SEQUENCE LISTING (6 sequences)

SEQ ID NO: 1 - DNA, 20 bp, Artificial Sequence
  Description of Artificial Sequence Synthetic primer
ttggttagcaagttaatacc

SEQ ID NO: 2 - DNA, 20 bp, Artificial Sequence
  Description of Artificial Sequence Synthetic primer
tagatttgaatgacttcccc

SEQ ID NO: 3 - protein, 314 aa, Artificial Sequence
  Description of Artificial Sequence Synthetic polypeptide
DRAMAQRKNAKSSGNSSSSGSGSGSTSAGSSSPGARRETKHGGHKNGRKGGLSGTSFFTWFMVIALLGVWTSVAVVWFDLVDYEEVLGKLGIYDADGDGDFDVDDAKVLLGLKERSTSEPAVPPEEAEPHTEPEEQVPVEAEPQNIEDEAKEQIQSLLHEMVHAEHVEGEDLQQEDGPTGEPQQEDDEFLMATDVDDRFETLEPEVSHEETEHSYHVEETVSQDCNQDMEEMMSEQENPDSSEPVVEDERLHHDTDDVTYQVYEEQAVYEPLENEGIEITEVTAPPEDNPVEDSQVIVEEVSIFPVEEQQEVPP

SEQ ID NO: 4 - protein, 176 aa, Artificial Sequence
  Description of Artificial Sequence Synthetic polypeptide
LDAAEKLRKRGKIEEAVNAFKELVRKYPQSPRARYGKAQCEDDLAEKRRSNEVLRGAIETYQEVASLPDVPADLLKLSLKRRSDRQQFLGHMRGSLLTLQRLVQLFPNDTSLKNDLGVGYLLIGDNDNAKKVYEEVLSVTPNDGFAKVHYGFILKAQNKIAESIPYLKEGIESGDP

SEQ ID NO: 5 - protein, 238 aa, Artificial Sequence
  Description of Artificial Sequence Synthetic polypeptide
GTDDGRFYFHLGDAMQRVGNKEAYKWYELGHKRGHFASVWQRSLYNVNGLKAQPWWTPKETGYTELVKSLERNWKLIRDEGLAVMDKAKGLFLPEDENLREKGDWSQFTLWQQGRRNENACKGAPKTCTLLEKFPETTGCRRGQIKYSIMHPGTHVWPHTGPTNCRLRMHLGLVIPKEGCKIRCANETRTWEEGKVLIFDDSFEHEVWQDASSFRLIFIVDVWHPELTPQQRRSLPAI

SEQ ID NO: 6 - protein, 9 aa, Artificial Sequence
  Description of Artificial Sequence Synthetic peptide
HEFMQAWET